(19) United States
(12) Patent Application Publication
(10) Pub. No.: US 2025/0025297 A1
(43) Pub. Date: Jan. 23, 2025

(54) DELIVERY APPARATUS FOR A DOCKING DEVICE

(71) Applicant: EDWARDS LIFESCIENCES CORPORATION, Irvine, CA (US)

(72) Inventor: Kurt Kelly Reed, Costa Mesa, CA (US)

(21) Appl. No.: 18/906,445

(22) Filed: Oct. 4, 2024

Related U.S. Application Data
(63) Continuation of application No. PCT/US2023/018453, filed on Apr. 13, 2023.
(60) Provisional application No. 63/362,996, filed on Apr. 14, 2022.

(51) Int. Cl. A61F 2/24

(52) U.S. Cl. CPC A61F 2/2427

(57) ABSTRACT

A handle assembly includes a first component having a male connection portion, which includes a main body with a pin extending radially outwardly from the main body. The handle assembly also includes a second component having a female connection portion configured to receive the male connection portion. The female connection portion has an inner surface defining an inner bore, a slot extending along the inner surface, and a retaining portion connected to the slot and configured to receive the pin. The first and second components are movable between a locked configuration and a release configuration by inserting the main body into the inner bore and rotating the male connection portion relative to the female connection portion less than one revolution about an axial axis of the main body. The pin is positioned within the retaining portion in the locked configuration and within the slot in the release configuration.

### CROSS-REFERENCE TO RELATED APPLICATIONS

This application is a continuation of PCT Patent Application No. PCT/US2023/018453, filed on Apr. 13, 2023, which claims the benefit of U.S. Provisional Application No. 63/362,996, filed Apr. 14, 2022, each of these applications being incorporated herein in its entirety by this specific reference.

### FIELD

The present disclosure concerns examples of delivery apparatuses for delivering a prosthetic implant into a patient's body and associated handle assemblies.

### BACKGROUND

Prosthetic valves can be used to treat cardiac valvular disorders. Native heart valves (e.g., the aortic, pulmonary, tricuspid and mitral valves) function to prevent backward flow or regurgitation, while allowing forward flow. These heart valves can be rendered less effective by congenital, inflammatory, infectious conditions, etc. Such conditions can eventually lead to serious cardiovascular compromise or death. For many years, the doctors attempted to treat such disorders with surgical repair or replacement of the valve during open heart surgery.

A transcatheter technique for introducing and implanting a prosthetic heart valve using a catheter in a manner that is less invasive than open heart surgery can reduce complications associated with open heart surgery. In this technique, a prosthetic valve can be mounted in a compressed state on the end portion of a catheter and advanced through a blood vessel of the patient until the valve reaches the implantation site. The valve at the catheter tip can then be expanded to its functional size at the site of the defective native valve, such as by inflating a balloon on which the valve is mounted or, for example, the valve can have a resilient, self-expanding stent or frame that expands the valve to its functional size when it is advanced from a delivery sheath at the distal end of the catheter. Optionally, the valve can have a balloon-expandable, self-expanding, mechanically expandable frame, and/or a frame expandable in multiple or a combination of ways.

In some instances, a transcatheter heart valve (THV) may be appropriately sized to be placed inside a particular native valve (e.g., a native aortic valve). As such, the THV may not be suitable for implantation at another native valve (e.g., a native mitral valve) and/or in a patient with a larger native valve. Additionally, or alternatively, the native tissue at the implantation site may not provide sufficient structure for the THV to be secured in place relative to the native tissue.

In certain instances, a docking device can be implanted first within the native valve and can be configured to receive a THV and secure (e.g., anchor) the THV in a desired position within the native valve. For example, the docking device can form a more circular and/or stable anchoring site at the native valve annulus in which a THV can be expanded and implanted. A transcatheter delivery apparatus can be used to deliver the docking device to the implantation site. In certain instances, the docking device can be arranged within the delivery apparatus, coaxial with additional components of the delivery apparatus.

Operating a delivery apparatus for implantation of a THV and/or a docking device involves complex steps and requires specialized skills. Accordingly, improvements to improvements to the transcatheter delivery apparatus to simplify its operation is desirable.

### SUMMARY

The present disclosure relates to devices and related methods for treating valvular regurgitation and/or other valve issues. Specifically, the present disclosure is directed to a delivery apparatus configured to deliver a prosthetic implant, such as a THV and/or a docking device, and the methods of implanting the prosthetic implant.

According to certain examples, a handle assembly for a delivery apparatus configured to deliver a prosthetic implant can include a first component having a male connection portion. The male connection portion can include a main body with a pin extending radially outwardly from the main body. The handle assembly can also include a second component having a female connection portion configured to receive the male connection portion of the first component. The female connection portion can have an inner surface defining an inner bore, a slot extending along the inner surface, and a retaining portion connected to the slot and configured to receive the pin. The first component and the second component can be movable between a locked configuration and a release configuration by inserting the main body into the inner bore and rotating the male connection portion relative to the female connection portion less than one revolution about an axial axis of the main body. In the locked configuration, the pin can be positioned within the retaining portion and the slot can be configured to resist rotational movement of male connection portion relative to the female connection portion. In the release configuration, the pin can be positioned within the slot.

According to certain examples, a handle assembly for a delivery apparatus configured to deliver a prosthetic implant can include a first component having a female connection portion. The female connection portion can have an inner surface defining an inner bore and a pin extending radially inwardly from the inner surface. The handle assembly can also include a second component having a male connection portion configured to be inserted into the inner bore of the female connection portion. The male connection portion can include an outer surface, a slot extending along the outer surface, and a retaining portion connected to the slot and configured to receive the pin. The first component and the second component can be movable between a locked configuration and a release configuration by inserting the male connection portion into the inner bore and rotating the male connection portion relative to the female connection portion less than one revolution about an axial axis of the inner bore. In the locked configuration, the pin can be positioned within the retaining portion and the slot can be configured to resist rotational movement of male connection portion relative to the female connection portion. In the release configuration, the pin can be positioned within the slot.

According to certain examples, a handle assembly for a delivery apparatus configured to deliver a prosthetic implant can include a suture lock configured to connect to a release suture which is tied to the prosthetic implant, and an adaptor having a lumen configured for the release suture to extend through and an end portion configured to be releasably connected to a connecting portion of the suture lock via a lock and release mechanism. The lock and release mechanism can include a peg and a slot configured to receive the peg. The slot can include a deflectable portion and a retaining portion connected to the deflectable portion. The retaining portion can be configured to retain the peg. The deflectable portion can have a smaller diameter than the peg. Rotation of the suture lock relative to the adaptor in a first direction can be configured to move the peg through the deflectable portion and lock into the retaining portion such that the deflectable portion inhibits the peg from moving out of the retaining portion. Rotation of the suture lock relative to the adaptor in a second direction opposite to the first direction can be configured to move the peg out of the retaining portion through the deflectable portion.

According to certain examples, a handle assembly for a delivery apparatus configured to deliver a prosthetic implant can include a suture lock configured to connect to a release suture which is tied to the prosthetic implant, and an adaptor having a lumen configured for the release suture to extend through and an end portion configured to be releasably connected to a connecting portion of the suture lock via a lock and release mechanism. When connected, the connecting portion of the suture lock and the end portion of the adaptor can be coaxial about an axial axis. The lock and release mechanism can include a peg and a slot configured to receive the peg. The peg can be movable between a compressed state and an expanded state, and the peg can be biased toward the expanded state. The slot can include a track portion and a retaining portion connected to the track portion. The retaining portion can be configured to retain the peg in the expanded state. The track portion can be configured to receive the peg in the compressed state and can have a diameter smaller than the peg in the expanded state. Rotation of the suture lock relative to the adaptor in a first direction can be configured to move the peg in the compressed state through the track portion and into the retaining portion such that the peg is locked therein in the expanded state and the track portion inhibits the peg from moving out of the retaining portion. Rotation of the suture lock relative to the adaptor in a second direction opposite to the first direction can be configured to move the peg from the expanded state to the compressed state and move the peg out of the retaining portion through the track portion.

According to certain examples, a handle assembly for a delivery apparatus configured to deliver a prosthetic implant can include a suture lock configured to connect to a release suture which is tied to the prosthetic implant, and an adaptor having a lumen configured for the release suture to extend through and an end portion configured to be releasably connected to a connecting portion of the suture lock via a lock and release mechanism. When connected, the connecting portion of the suture lock and the end portion of the adaptor can be coaxial about an axial axis. The lock and release mechanism can include a peg and a slot configured to receive the peg. The slot can include an axial portion extending parallel to the axial axis and a circumferential portion connected to the axial portion and extending circumferentially about the axial axis. The lock and release mechanism can further include a retaining portion connected to a terminal end of the circumferential portion and configured to retain the peg. The retaining portion can have a larger diameter than the circumferential portion. Rotation of the suture lock relative to the adaptor in a first direction can be configured to move the peg through the circumferential portion and locked into the retaining portion such that the circumferential portion inhibits the peg from moving out of the retaining portion. Rotation of the suture lock relative to the adaptor in a second direction opposite to the first direction can be configured to move the peg out of the retaining portion through the circumferential portion.

According to certain examples, a handle assembly for a delivery apparatus configured to deliver a prosthetic implant can include a suture lock configured to connect to a release suture which is tied to the prosthetic implant, and an adaptor having a lumen configured for the release suture to extend through and an end portion configured to be releasably connected to a connecting portion of the suture lock via a lock and release mechanism. When connected, the connecting portion of the suture lock and the end portion of the adaptor can be coaxial about an axial axis. The lock and release mechanism can include a first locking mechanism and a second locking mechanism. The first locking mechanism can include a first peg and a first slot configured to receive the first peg. When the first peg is received within a circumferential portion of the first slot, the suture lock can be configured to be rotatable but not axially movable relative to the adaptor. The second locking mechanism can include a second peg and a second slot configured to receive the second peg. The second slot can extend circumferentially about the axial axis. A radially recessed detent can be connected to the second slot and configured to retain the second peg. When the first peg is received within the circumferential portion of the first slot, rotation of the suture lock relative to the adaptor in a first direction can be configured to move the second peg through the second slot until the second peg snap fits into the radially recessed detent such that the second slot inhibits the second peg from moving out of the radially recessed detent. Rotation of the suture lock relative to the adaptor in a second direction opposite to the first direction can be configured to move the second peg out of the radially recessed detent through the second slot.

Certain examples of the disclosure also concern a delivery apparatus configured to deliver a prosthetic implant. The delivery apparatus can include any one of the handle assemblies described above and a delivery sheath extending distally from the handle assembly.

Certain examples of the disclosure also concern a method for implanting a prosthetic implant. The method can include assembling any one of the handle assemblies described above. The assembling can include rotating the suture lock relative to the adaptor in the first direction so as to connect the suture lock to the adaptor.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 schematically illustrates a docking device delivery apparatus implanting a docking device for a prosthetic heart valve at a mitral valve of a patient, according to one example.

FIG. 2A schematically illustrates the docking device of FIG. 1 fully implanted at the mitral valve of the patient after the docking device delivery apparatus has been removed from the patient.

FIG. 2B schematically illustrates a prosthetic heart valve delivery apparatus implanting a prosthetic heart valve in the implanted docking device of FIG. 2A at the mitral valve of the patient, according to one example.

FIG. 3 is a side perspective view of a docking device in a helical configuration, according to one example.

FIG. 3A is a cross-sectional view of the docking device of FIG. 3 taken along line 3A-3A, according to one example.

FIG. 3B is a cross-sectional view of the docking device taken along the same line as in FIG. 3A, except in FIG. 3B, the docking device is in a substantially straight delivery configuration.

FIG. 3C is a cross-sectional view of the docking device of FIG. 3 taken along line 3A-3A, according to another example.

FIG. 3D is a cross-sectional view of the docking device taken along the same line as in FIG. 3C, except in FIG. 3D, the docking device is in a substantially straight delivery configuration.

FIG. 4 is a side view of an exemplary delivery apparatus for a docking device, the delivery apparatus including a handle assembly and an outer shaft extending distally from the handle assembly.

FIG. 5 is a side view of a hub assembly of the handle assembly of the delivery apparatus of FIG. 4.

FIG. 6 is a perspective view of a distal end portion of the delivery apparatus of FIG. 4, which illustrates an exemplary docking device deployed from an outer shaft of the delivery apparatus and covered by a sleeve shaft of the delivery apparatus.

FIG. 7 is a perspective view of a distal end portion of the delivery apparatus of FIG. 4, which illustrates the exemplary docking device of FIG. 10 deployed from the outer shaft of the delivery apparatus with the sleeve shaft removed from the docking device.

FIG. 8A depicts a portion of a handle assembly for a delivery apparatus including a suture lock and a sleeve handle, according to one example.

FIG. 8B is a perspective view of the suture lock of FIG. 8A, disconnected from a branch of the handle assembly.

FIG. 8C is an exploded view of the suture lock of FIG. 8A.

FIG. 9A is a side view of the suture lock of FIGS. 8A-8C including a flushing port at a proximal end of the suture lock.

FIG. 9B is a perspective view of a detail portion of the suture lock of FIGS. 8A-8C, showing a release knob and an internal release bar.

FIG. 9C is a side cross-sectional view of the release bar of the suture lock of FIG. 9B.

FIG. 9D is a perspective view of the release bar of FIGS. 9B-9C.

FIG. 9E is a detail cross-sectional view of a suture cutting portion of the release bar of FIGS. 9B-9D.

FIG. 10A depicts a suture lock connected to an adaptor of a handle assembly, according to one example.

FIG. 10B depicts the suture lock is partially rotated relative to the adaptor of FIG. 10A.

FIG. 10C depicts the suture lock disconnected from the adaptor of FIG. 10A.

FIG. 10D depicts a lock pin configured to use together with the suture lock of FIG. 10A.

FIG. 11A is a top view of an example lock and release mechanism through which a suture lock can be connected to and/or disconnected from an adaptor.

FIG. 11B is a side view of the lock and release mechanism of FIG. 11A.

FIG. 12A is a side view of another example lock and release mechanism through which a suture lock can be connected to and/or disconnected from an adaptor.

FIG. 12B is a side view of the lock and release mechanism of FIG. 12A.

FIG. 13 is a top view of another example lock and release mechanism through which a suture lock can be connected to and/or disconnected from an adaptor, the lock and release mechanism comprising an axial locking mechanism and a rotational locking mechanism.

FIG. 14A is a cross-sectional view of the rotational locking mechanism of FIG. 13, according to one example.

FIG. 14B is a cross-sectional view of the rotational locking mechanism of FIG. 13, according to another example.

FIG. 15 is a top view of a lock and release mechanism through which a suture lock can be connected to and/or disconnected from an adaptor, according to yet another example.

### DETAILED DESCRIPTION

### General Considerations

It should be understood that the disclosed examples can be adapted to deliver and implant prosthetic devices in any of the native annuluses of the heart (e.g., the pulmonary, mitral, and tricuspid annuluses), and can be used with any of various delivery approaches (e.g., retrograde, antegrade, transseptal, transventricular, transatrial, etc.).

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the term “approximately” and “about” means the listed value and any value that is within 10% of the listed value. For example, “about 1 mm” means any value between about 0.9 mm and about 1.1 mm, inclusive.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

### Exemplary Transcatheter Heart Valve Replacement Procedure

Described herein are various systems, apparatuses, methods, or the like, that can be used in or with delivery apparatuses to deliver a prosthetic implant (e.g., a prosthetic valve, a docking device, etc.) into a patient body.

In certain examples, a delivery apparatus can be configured to deliver and implant a docking device at an implantation site, such as a native valve annulus. The docking device can be configured to more securely hold an expandable prosthetic valve implanted within the docking device, at the native valve annulus. For example, a docking device can provide or form a more circular and/or stable anchoring site, landing zone, or implantation zone at the implant site, in which a prosthetic valve can be expanded or otherwise implanted. By providing such anchoring or docking devices, replacement prosthetic valves can be more securely implanted and held at various valve annuluses, including at the mitral annulus which does not have a naturally circular cross-section.

In some examples, the docking device can be arranged within an outer shaft of the delivery apparatus. A sleeve shaft can cover or surround the docking device within the delivery apparatus and during delivery to a target implantation site. A pusher shaft can be disposed within the outer shaft, proximal to the docking device, and configured to push the docking device out of the outer shaft to position the docking device at the target implantation site. The sleeve shaft can also surround the pusher shaft within the outer shaft of the delivery apparatus. After positioning the docking device at the target implantation site, the sleeve shaft can be removed from the docking device and retracted back into the outer shaft of the delivery apparatus.

Fluid (e.g., a flush fluid, such as heparinized saline or the like) can be provided to a pusher shaft lumen defined within an interior of the pusher shaft and a delivery shaft lumen defined between the sleeve shaft and the outer shaft of the delivery apparatus. Fluid from the pusher shaft lumen can then flow to a sleeve shaft lumen defined between the docking device and the sleeve shaft and the sleeve shaft and the pusher shaft. By providing a consistent flow of fluid through these lumens of the delivery apparatus, stagnation of blood within the delivery apparatus can be reduced or avoided, thereby decreasing or preventing thrombus formation.

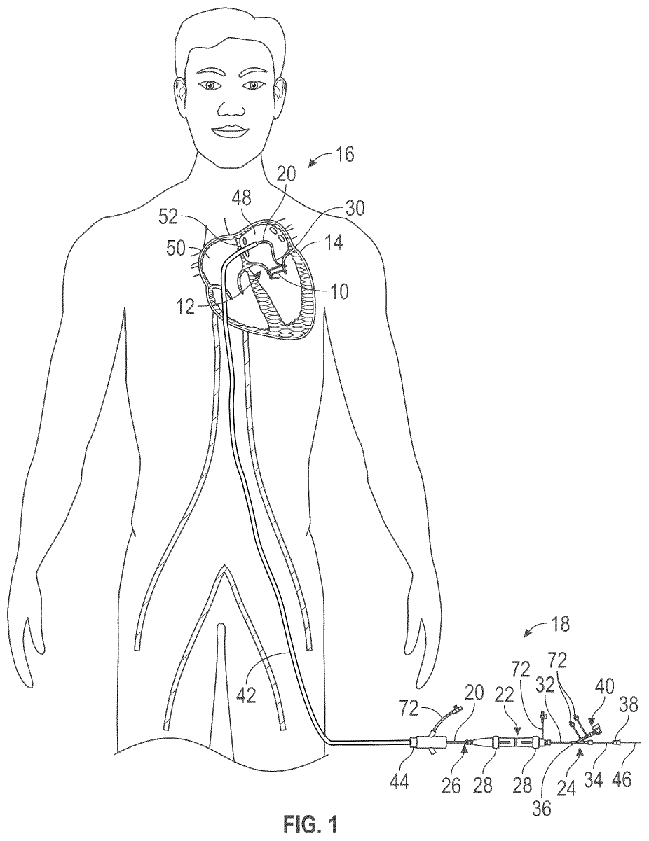

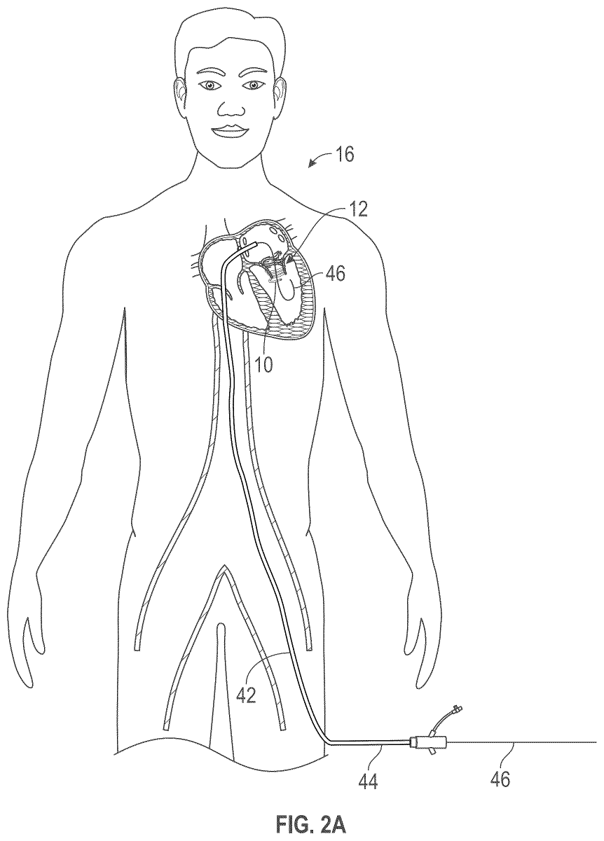

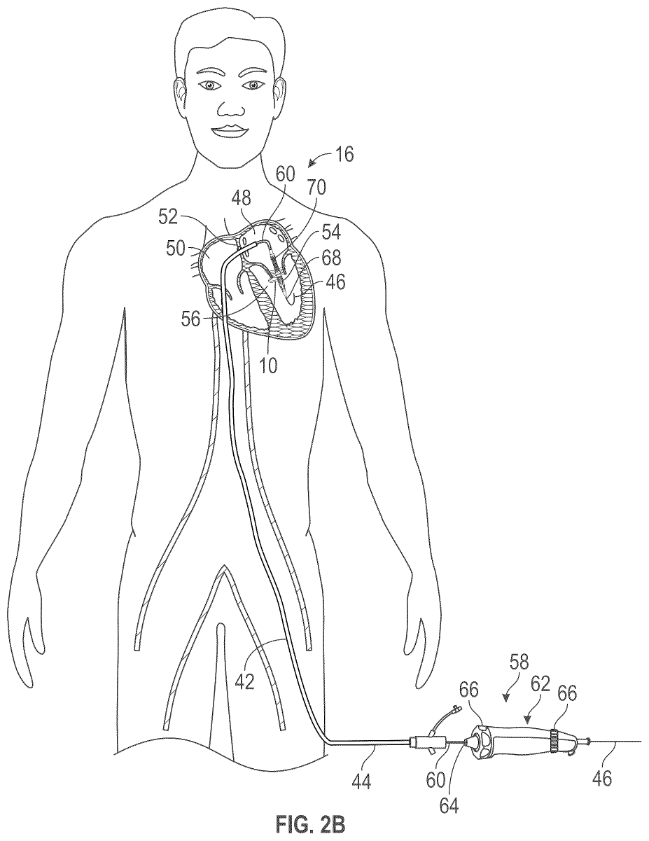

An exemplary transcatheter heart valve replacement procedure which utilizes a first exemplary delivery apparatus to deliver a docking device to a native valve annulus and then a second exemplary delivery apparatus to deliver a prosthetic heart valve (e.g., THV) inside the docking device is depicted in the schematic illustrations of FIGS. 1 and 2A-2B.

As introduced above, defective native heart valves may be replaced with THVs. However, in certain instances, such THVs may not be able to sufficiently secure themselves to the native tissue (e.g., to the leaflets and/or annulus of the native heart valve) and may undesirably shift around relative to the native tissue, leading to paravalvular leakage, valve malfunction, and/or other issues. Thus, a docking device may be implanted first at the native valve annulus and then the THV can be implanted within the docking device to help anchor the THV to the native tissue and provide a seal between the native tissue and the THV.

FIGS. 1 and 2A-2B depict an exemplary transcatheter heart valve replacement procedure which utilizes a docking device. During the procedure, a user first delivers and implants the docking device at a patient's native heart valve using a docking device delivery apparatus (see, e.g., FIG. 1), then removes the docking device delivery apparatus from the patient after implanting the docking device (see, e.g., FIG. 2A), and finally implants the THV within the implanted docking device using a prosthetic valve delivery apparatus (see, e.g., FIG. 2B).

FIG. 1 depicts a first stage in an exemplary mitral valve replacement procedure where a docking device 10 is being implanted at a mitral valve 12 of a heart 14 of a patient 16 using a docking device delivery apparatus 18 (which may also be referred to herein as “dock delivery catheter,” “dock delivery system,” or simply “delivery system”).

In general, the docking device delivery apparatus 18 comprises a delivery shaft 20, a handle 22, and a pusher assembly 24. The delivery shaft 20 can be configured to extend into the patient's vasculature and provide a passageway for the docking device 10 to reach the target implantation site (e.g., mitral valve 12). Specifically, a user can advance the delivery shaft 20 (which is configured to receive and/or retain the docking device 10 therein) through the patient's vasculature to the implantation site. In some examples, the delivery shaft 20 can comprise an outer sheath or shaft that defines a lumen, and the pusher assembly 24 and/or docking device 10 can be configured to be received and/or advanced within such lumen.

The handle 22 is configured to be gripped and/or otherwise held by the user to advance the delivery shaft 20 through the patient's vasculature. Specifically, the handle 22 can be coupled to a proximal end 26 of the delivery shaft 20 and can be configured to remain accessible to the user (e.g., outside the patient 16) during the docking device implantation procedure. In this way, the user can advance the delivery shaft 20 through the patient's vasculature by exerting a force on (e.g., pushing) the handle 22. In some examples, the delivery shaft 20 can be configured to carry the pusher assembly 24 and/or the docking device 10 with it as it advances through the patient's vasculature. In this way, the docking device 10 and/or the pusher assembly 24 can advance through the patient's vasculature in lockstep with the delivery shaft 20 as the user grips the handle 22 and pushes the delivery shaft 20 deeper into the patient's vasculature.

In some examples, the handle 22 can comprise one or more articulation members 28 that are configured to aid in navigating the delivery shaft 20 through the patient's vasculature. Specifically, the articulation members 28 can comprise one or more of knobs, buttons, wheels, and/or other types of physically adjustable control members that are configured to be adjusted by the user to flex, bend, twist, turn, and/or otherwise articulate a distal end 30 of the delivery shaft 20 to aid in navigating the delivery shaft 20 through the patient's vasculature.

The pusher assembly 24 can be configured to deploy and/or implant the docking device 10 at the implantation site (e.g., native valve). Specifically, the pusher assembly 24 can be configured to be adjusted by the user to advance the docking device 10 through the delivery shaft 20 and push the docking device 10 out of the distal end 30 of the delivery shaft 20. As described above, the pusher assembly 24 can be configured to extend through the delivery shaft 20, within the lumen defined by the outer sheath of the delivery shaft 20. The pusher assembly 24 also can be coupled to the docking device 10 such that the pusher assembly 24 can push the docking device 10 through and/or out of the delivery shaft 20 as the pusher assembly 24 advances through the delivery shaft 20. Stated slightly differently, because it is retained by, held, and/or otherwise coupled to the pusher assembly 24, the docking device 10 can advance in lockstep with the pusher assembly 24 through and/or out of the delivery shaft 20.

The pusher assembly 24 can include a pusher shaft 32 and, in some examples, can also include a sleeve shaft 34. The pusher shaft 32 can be configured to advance the docking device 10 through the delivery shaft 20 and out of the distal end 30 of the delivery shaft 20, while the sleeve shaft 34, when included, can have a distal section configured to cover the docking device 10 within the delivery shaft 20 and while pushing the docking device 10 out of the delivery shaft 20 and positioning the docking device 10 at the implantation site. In some examples, the pusher shaft 32 can be covered by the sleeve shaft 34 and arranged within an outer shaft or connector of a pusher handle (also referred to herein as a “hub assembly”) 36, as described further below.

In some examples, the pusher assembly 24 can comprise the pusher handle 36 that is coupled to the pusher shaft 32 and that is configured to be gripped and pushed by the user to translate the pusher shaft 32 axially relative to the delivery shaft 20 (e.g., to push the pusher shaft 32 into and/or out of the distal end 30 of the delivery shaft 20). The sleeve shaft 34 can be configured to be retracted and/or withdrawn from the docking device 10, after positioning the docking device 10 at the target implantation site. For example, the pusher assembly 24 can include a sleeve handle 38 that is coupled to the sleeve shaft 34 and is configured to be pulled by a user to retract (e.g., axially move) the sleeve shaft 34 relative to the pusher shaft 32.

The pusher assembly 24 can be removably coupled to the docking device 10, and as such can be configured to release, detach, decouple, and/or otherwise disconnect from the docking device 10 once the docking device 10 has been deployed at the target implantation site. As just one example, the pusher assembly 24 (e.g., pusher shaft 32) may be removably coupled to the docking device 10 via a thread, string, yarn, suture, or other suitable material that is tied or sutured to the docking device 10.

In some examples, the pusher assembly 24 can include a suture lock assembly 40 (also referred to as a “suture lock”) that is configured to receive and/or hold the thread or other suitable material that is coupled to the docking device 10 via a suture. The thread or other suitable material that forms the suture can extend from the docking device 10, through the pusher assembly 24, to the suture lock assembly 40. The suture lock assembly 40 can also be configured to cut the suture to release, detach, decouple, and/or otherwise disconnect the docking device 10 from the pusher assembly 24. For example, the suture lock assembly 40 can comprise a cutting mechanism that is configured to be adjusted by the user to cut the suture.

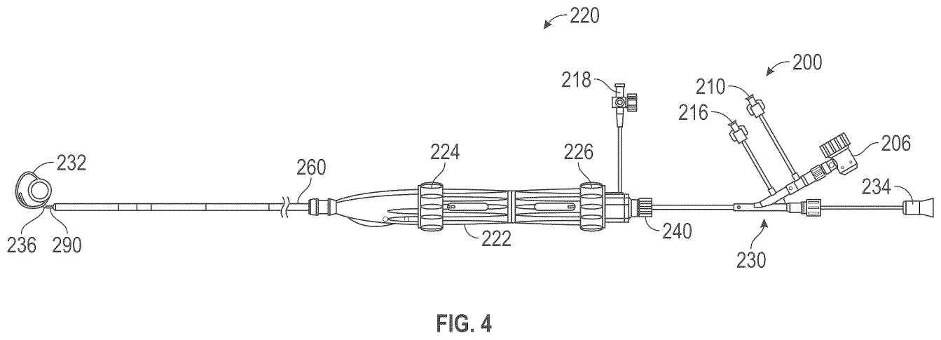

Further details of the docking device delivery apparatus and its variants are described further below with reference to FIGS. 4-15 and are described in International Application No. PCT/US2020/36577, which is incorporated by reference herein in its entirety.

Before inserting the docking device delivery apparatus 18 into the vasculature of the patient 16, the user may first make an incision in the patient's body to access a blood vessel 42. For example, as illustrated in FIG. 1, the user may make an incision in the patient's groin to access a femoral vein. Thus, in certain examples, the blood vessel 42 may be a femoral vein.

After making the incision at the blood vessel 42, the user may insert an introducer device 44, a guidewire 46, and/or other devices (e.g., delivery shaft 20, pusher shaft 32, and/or sleeve shaft 34 of the docking device delivery apparatus 18, catheters, and/or other delivery apparatuses, docking device 10, prosthetic valves, etc.) through the incision and into the blood vessel 42. The introducer device 44 (which can include an introducer sheath) can be configured to facilitate the percutaneous introduction of the guidewire 46 and/or the other devices (e.g., docking device delivery apparatus 18) into and through the blood vessel 42 and can extend through only a portion of the blood vessel 42 even when it is fully inserted by the user (i.e., it may extend through the blood vessel 42 towards the heart 14, but may stop short of the heart 14). The guidewire 46 on the other hand, can be configured to guide the delivery apparatuses (e.g., docking device delivery apparatus 18, prosthetic valve delivery apparatuses, catheters, etc.) and their associated devices (e.g., docking device, prosthetic heart valve, etc.) to the target implantation site, and thus may extend all the way through the blood vessel 42 and into a left atrium 48 of the heart 14. Specifically, the user may advance the guidewire 46 through the blood vessel 42 (e.g., through the femoral vein and inferior vena cava) to a right atrium 50 of the heart 14. The user may make a small incision in an atrial septum 52 of the heart 14 to allow the guidewire 46 to pass from the right atrium 50 to the left atrium 48 of the heart 14 and may then advance the guidewire 46 through the incision in the atrial septum 52 into the left atrium 48. Thus, the guidewire 46 can provide a pathway that the docking device delivery apparatus 18 can follow as it advances through the patient's vasculature to ensure that the docking device delivery apparatus 18 does not perforate the walls of the blood vessel 42 and/or other vasculature tissue.

After positioning the guidewire 46 within the left atrium 48, the user can insert the docking device delivery apparatus 18 (e.g., delivery shaft 20) into the patient 16 by advancing the docking device delivery apparatus 18 through the introducer device 44 and over the guidewire 46. The user can then continue to advance the docking device delivery apparatus 18 through the patient's vasculature along the guidewire 46 until the docking device delivery apparatus 18 reaches the left atrium 48, as illustrated in FIG. 1. Specifically, the user can advance the delivery shaft 20 of the docking device delivery apparatus 18 by gripping and exerting a force on (e.g., pushing) the handle 22 of the docking device delivery apparatus 18. While advancing the delivery shaft 20 through the patient's vasculature, the user can adjust the one or more articulation members 28 of the handle 22 to navigate the various turns, corners, constrictions, and/or other obstacles in the patient's vasculature.

After the delivery shaft 20 reaches the left atrium 48, the user can position the distal end 30 of the delivery shaft 20 at and/or near the posteromedial commissure of the mitral valve 12 using the handle 22 (e.g., the articulation members 28). The user can then push the docking device 10 out of the distal end 30 of the delivery shaft 20 with the pusher assembly 24 to deploy and/or implant the docking device 10 at the mitral valve 12. For example, the user can actuate the pusher handle 36 to axially translate the pusher shaft 32, in a distal direction, relative to the delivery shaft 20, such that the docking device 10 (which can be covered by the sleeve shaft 34) is deployed out of the delivery shaft 20 and moved into a desired position at the target implantation site.

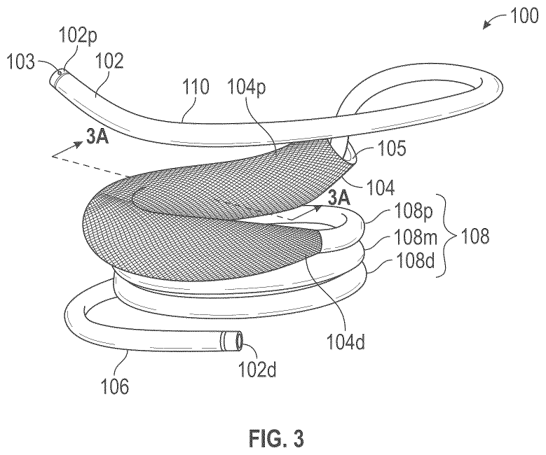

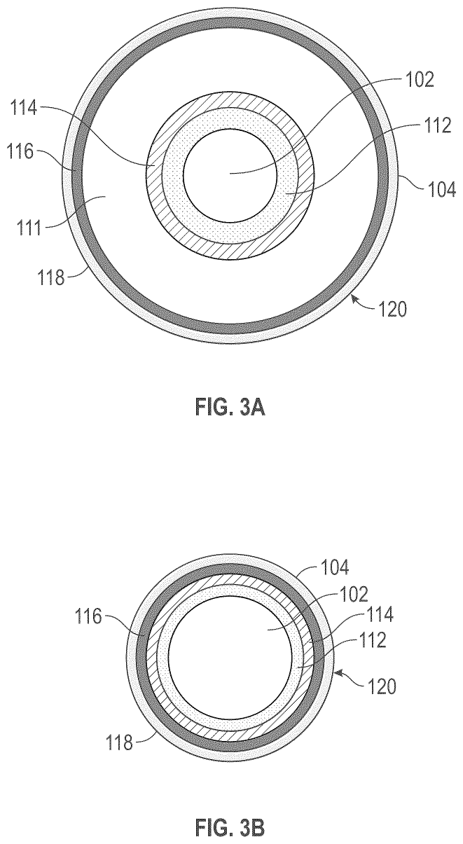

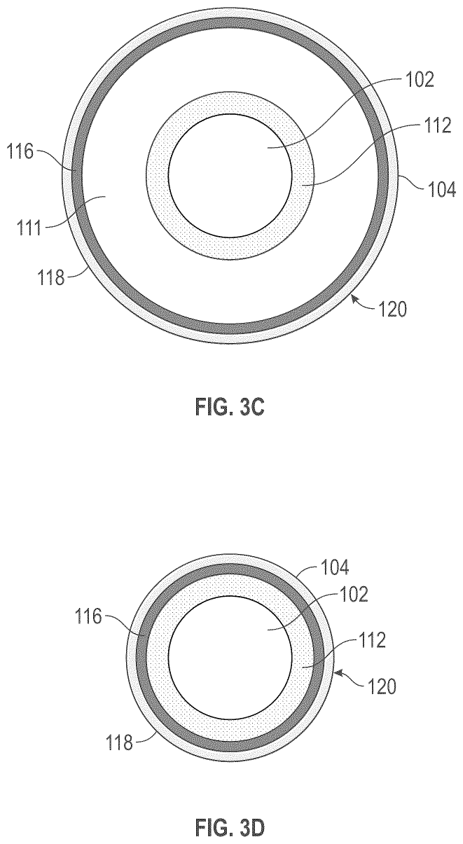

In some examples, the docking device 10 can be constructed from, formed of, and/or comprise a shape memory material, and as such, may return to its original, pre-formed shape when it exits the delivery shaft 20 and is no longer constrained by the delivery shaft 20. As one example, the docking device 10 may originally be formed as a coil, and thus may wrap around the ventricular side of the native leaflets as it exits the delivery shaft 20 and returns to its original coiled configuration (e.g., as shown in FIG. 3 and described further below).

After pushing the ventricular portion of the docking device 10 (i.e., the portion of the docking device 10 that is configured to be positioned/disposed within a left ventricle 56 and/or on the ventricular side of the mitral valve leaflets), the user can then release the remaining portion of the docking device 10 (the atrial portion of the docking device 10) from the delivery shaft 20 within the left atrium 48. Specifically, the user can retract the delivery shaft 20 relative to the docking device 10, away from the posteromedial commissure of the mitral valve 12. In some examples, the user can maintain the position of the pusher shaft 32 (e.g., by exerting a holding and/or pushing force on the pusher shaft 32) while retracting the delivery shaft 20 proximally so that the delivery shaft 20 withdraws and/or otherwise retracts relative to the docking device 10 and the pusher shaft 32. In this way, the pusher shaft 32 can hold the docking device 10 in place while the user retracts the delivery shaft 20, thereby releasing the docking device 10 from the delivery shaft 20. In some examples, the user can also retract the sleeve shaft 34 from the docking device 10 to uncover the docking device 10. In some examples, the user can also deploy an expandable sleeve of the docking device 10.

After deploying and/or implanting the docking device 10, the user can decouple and/or otherwise disconnect the docking device delivery apparatus 18 from the docking device 10 by, for example, cutting the suture that is tied to the docking device 10. As just one example, the user may cut the suture with the cutting mechanism of the suture lock assembly 40. Once the docking device 10 is disconnected from the docking device delivery apparatus 18, the user can retract the entire docking device delivery apparatus 18 (e.g., the delivery shaft 20, handle 22, and pusher assembly 24) from the patient 16 so that the user can deliver and implant the THV at the mitral valve 12. In one example, the docking device 10 and the THV may be delivered on two different, separate delivery apparatuses, and thus the user may need to remove the docking device delivery apparatus 18 from the patient 16 to make room for the THV delivery apparatus. As another example, the user may need to remove the docking device delivery apparatus 18 from the patient 16 to load the THV onto the same delivery apparatus. In either example, the user may need to remove the docking device delivery apparatus 18 from the patient 16 before implanting the THV.

FIG. 2A depicts a second stage in the mitral valve replacement procedure, where the docking device 10 has been fully deployed and implanted at the mitral valve 12. The docking device delivery apparatus 18 (including the delivery shaft 20) has been removed from the patient 16 such that only the guidewire 46 and the introducer device 44 remain inside the patient 16. The introducer device 44 may remain inside the patient 16 to help percutaneously inserting the THV and the prosthetic valve delivery apparatus into the patient 16, while the guidewire 46 may remain within the patient's vasculature to help advance the THV and the prosthetic valve delivery apparatus through the patient's vasculature. Specifically, the guidewire 46 may ensure that the THV and the prosthetic valve delivery apparatus do not perforate the walls of the blood vessel 42 and/or other vasculature tissue as they advance through the patient's vasculature. In some examples, the user may advance the guidewire 46 through the mitral valve 12 and into the left ventricle 56 to ensure that the guidewire 46 routes the THV and the prosthetic valve delivery apparatus all of the way to the mitral valve 12 and into the docking device 10.

As illustrated in FIG. 2A, the docking device 10 can be configured to wrap around the ventricular side of the leaflets of the mitral valve 12 and squeeze the leaflets radially inwardly (i.e., radially compress the leaflets) to adjust the size and/or shape of the opening between the two leaflets of the mitral valve 12. For example, the docking device 10 can be configured to reduce the size of the opening of the mitral valve 12 and/or to change the shape of the opening to more closely match the cross-sectional shape and/or profile of the THV (e.g., make the opening more circular for a cylindrical THV). By constricting the mitral valve 12 in this manner, the docking device 10 can provide a tighter fit, and thus a better seal, between the THV and the mitral valve 12.

FIG. 2B depicts a third stage in the mitral valve replacement procedure where the user delivers and/or implants a prosthetic heart valve 54 within the docking device 10 and/or at the mitral valve 12 using a prosthetic heart valve delivery apparatus 58. Thus, the docking device 10 and the prosthetic heart valve 54 can be delivered on different delivery apparatuses at different stages in the mitral valve replacement procedure. Specifically, the docking device 10 can be delivered to the mitral valve 12 with the docking device delivery apparatus 18 during the first stage of the mitral valve replacement procedure and the prosthetic heart valve 54 can then be delivered with the prosthetic heart valve delivery apparatus 58.

The prosthetic heart valve delivery apparatus 58 comprises a delivery shaft 60 and a handle 62 coupled to a proximal end 64 of the delivery shaft 60. The delivery shaft 60 can be configured to extend into the patient's vasculature to deliver, implant, expand, and/or otherwise deploy the prosthetic heart valve 54 within the docking device 10 at the mitral valve 12. The handle 62 can be the same as, or similar to, the handle 22 of the docking device delivery apparatus 18 and can be similarly configured to be gripped and/or otherwise held by the user to advance the delivery shaft 60 through the patient's vasculature.

In some examples, the handle 62 can comprise one or more articulation members 66 that are configured to aid in navigating the delivery shaft 60 through the patient's vasculature. Specifically, the articulation members 66 can comprise one or more of knobs, buttons, wheels, and/or other types of physically adjustable control members that are configured to be adjusted by the user to flex, bend, twist, turn, and/or otherwise articulate a distal end 68 of the delivery shaft 60 to aid in navigating the delivery shaft 60 through the patient's vasculature.

In some examples, the prosthetic heart valve delivery apparatus 58 can comprise an expansion mechanism 70 that is configured to radially expand and deploy the prosthetic heart valve 54. For example, the expansion mechanism 70 can comprise an inflatable balloon that is configured to be inflated to radially expand the prosthetic heart valve 54 within the docking device 10. The expansion mechanism 70 can be included in and/or coupled to the delivery shaft 60 at and/or proximate to the distal end 68 of the delivery shaft 60. In other examples, the prosthetic heart valve 54 can be self-expanding and can be configured to radially expand on its own without the expansion mechanism 70. In other examples, the prosthetic heart valve 54 can be mechanically expandable and the prosthetic heart valve delivery apparatus 58 can include one or more mechanical actuators configured to radially expand the prosthetic heart valve 54.

Additional details of the prosthetic heart valve 54 and its components are described, for example, in U.S. Patent Nos. 9,393, 110, 9,339,384, 9,155,619, 8,652,202, and 6,730,118, U.S. Patent Publication Nos. 2019/0374337, 2019/0192296, 2019/0046314, 2018/0153689, and PCT Patent Application Publication Nos. WO/2021/188476 and WO/2020/247907, the disclosures of which are incorporated herein in their entireties for all purposes.

Prosthetic heart valve 54 can be coupled to the delivery shaft 60 at and/or proximate to the distal end 68 of the delivery shaft 60. In certain examples where the prosthetic heart valve delivery apparatus 58 includes the expansion mechanism 70, the prosthetic heart valve 54 can be mounted on the expansion mechanism 70 in a radially compressed configuration. In some examples, the prosthetic heart valve 54 can be removably coupled to the delivery shaft 60 such that, after the prosthetic heart valve 54 is radially expanded and deployed from the prosthetic heart valve delivery apparatus 58, the prosthetic heart valve delivery apparatus 58 can be retracted away from the implanted prosthetic heart valve 54 and removed from the patient 16.

Prosthetic heart valve 54 can be configured to be received and/or retained within the docking device 10. That is, docking device 10 can be configured to receive the prosthetic heart valve 54 and help anchor the prosthetic heart valve 54 to the mitral valve 12. As will be explained in further detail below, the docking device 10 can also be configured to provide a seal between the prosthetic heart valve 54 and the leaflets of the mitral valve to reduce paravalvular leakage around the prosthetic heart valve 54. Specifically, as introduced above, the docking device 10 can initially constrict the leaflets of the mitral valve 12. The prosthetic heart valve 54 can then push the leaflets against the docking device 10 as it radially expands within the docking device 10 (e.g., via inflation of the expansion mechanism 70). Thus, the docking device 10 and the prosthetic heart valve 54 can be configured to sandwich the leaflets of the mitral valve 12 when the prosthetic heart valve 54 is expanded within the docking device 10. In this way, the docking device 10 can provide a seal between the leaflets of the mitral valve 12 and the prosthetic heart valve 54.

In some examples, one or more of the docking device delivery apparatus 18, the prosthetic heart valve delivery apparatus 58, and/or the introducer device 44 can comprise one or more flushing ports 72 (see, e.g., FIG. 1) that are configured to supply flushing fluid to the lumens thereof (e.g., lumens of the delivery shaft 20 of docking device delivery apparatus 18, the delivery shaft 60 of the prosthetic heart valve delivery apparatus 58, and/or the introducer device 44) to prevent and/or reduce the likelihood of blood clot (e.g., thrombus) formation.

Like when delivering the docking device 10, the user can insert the prosthetic heart valve delivery apparatus 58 (e.g., delivery shaft 60) into the patient 16 by advancing the prosthetic heart valve delivery apparatus 58 through the introducer device 44 and over the guidewire 46. The user can continue to advance the prosthetic heart valve delivery apparatus 58 along the guidewire 46 and through the patient's vasculature until the prosthetic heart valve delivery apparatus 58 reaches the mitral valve 12, as illustrated in FIG. 2B. Specifically, the user can advance the delivery shaft 60 of the prosthetic heart valve delivery apparatus 58 by gripping and exerting a force on (e.g., pushing) the handle 62 of the prosthetic heart valve delivery apparatus 58. While advancing the delivery shaft 60 through the patient's vasculature, the user can adjust the one or more articulation members 66 of the handle 62 to navigate the various turns, corners, constrictions, and/or other obstacles in the patient's vasculature.

The user can advance the delivery shaft 60 along the guidewire 46 until the prosthetic heart valve 54 and/or the expansion mechanism 70 is/are positioned/disposed within the docking device 10 and/or the mitral valve 12. For example, the user may advance the delivery shaft 60 along the guidewire 46 until the delivery shaft 60 extends through the mitral valve 12, such that the distal end 68 of the delivery shaft 60 is positioned/disposed within the left ventricle 56. After the prosthetic heart valve 54 is appropriately positioned/disposed within the docking device 10, the user can radially expand the prosthetic heart valve 54, e.g., with the expansion mechanism 70, to its fully expanded position or configuration. In some examples, the user can lock the prosthetic heart valve 54 in its fully expanded position (e.g., with a locking mechanism) to prevent the valve from collapsing. After expanding and deploying the prosthetic heart valve 54, the user can decouple and/or otherwise disconnect the delivery shaft 60 from the prosthetic heart valve 54 and remove the delivery shaft 60 from the patient.

Additional examples of the dock delivery system and methods of implanting a docking device and implanting a prosthetic valve within the docking device are described in International Patent Application PCT/US2021/056150, which is incorporated by reference herein in its entirety.

Although FIGS. 1 and 2A-2B specifically depict a mitral valve replacement procedure, it should be appreciated that the same and/or similar procedure may be utilized to replace other heart valves (e.g., tricuspid, pulmonary, and/or aortic valves). Further, the same and/or similar delivery apparatuses (e.g., docking device delivery apparatus 18, prosthetic heart valve delivery apparatus 58, introducer device 44, and/or guidewire 46), docking devices (e.g., docking device 10), replacement heart valves (e.g., prosthetic heart valve 54), and/or components thereof may be utilized for replacing these other heart valves.

For example, when replacing a native tricuspid valve, the user may also access the right atrium 50 via a femoral vein but may not need to cross the atrial septum 52 into the left atrium 48. Instead, the user may leave the guidewire 46 in the right atrium 50 and perform the same and/or similar docking device implantation process at the tricuspid valve. Specifically, the user may push the docking device 10 out of the delivery shaft 20 around the ventricular side of the tricuspid valve leaflets, release the remaining portion of the docking device 10 from the delivery shaft 20 within the right atrium 50, and then remove the delivery shaft 20 of the docking device delivery apparatus 18 from the patient 16. The user may then advance the guidewire 46 through the tricuspid valve into the right ventricle and perform the same and/or similar prosthetic heart valve implantation process at the tricuspid valve, within the docking device 10. Specifically, the user may advance the delivery shaft 60 of the prosthetic heart valve delivery apparatus 58 through the patient's vasculature along the guidewire 46 until the prosthetic heart valve 54 is positioned/disposed within the docking device 10 and the tricuspid valve. The user may then expand the prosthetic heart valve 54 within the docking device 10 before removing the prosthetic heart valve delivery apparatus 58 from the patient 16. In another example, the user may perform the same and/or similar process to replace the aortic valve but may access the aortic valve from the outflow side of the aortic valve via a femoral artery.

Further, although FIGS. 1 and 2A-2B depict a mitral valve replacement procedure that accesses the mitral valve 12 from the left atrium 48 via the right atrium 50 and femoral vein, it should be appreciated that the mitral valve 12 may alternatively be accessed from the left ventricle 56. For example, the user may access the mitral valve 12 from the left ventricle 56 via the aortic valve by advancing one or more delivery apparatuses through an artery to the aortic valve, and then through the aortic valve into the left ventricle 56.

### Exemplary Docking Device

FIG. 3 shows an example of a docking device 100 configured to receive a prosthetic heart valve. For example, the docking device 100 can be implanted within a native valve annulus, as described above with reference to FIGS. 1 and 2A. As depicted in FIGS. 1 and 2A-2B, the docking device 100 can be configured to receive and secure a prosthetic valve within the docking device, thereby securing the prosthetic valve at the native valve annulus.

Referring to FIG. 3, the docking device 100 can comprise two main components: a coil 102 and a guard member 104 covering at least a portion of the coil 102. In certain examples, the coil 102 can include a shape memory material (e.g., Nitinol) such that the docking device 100 (and the coil 102) can move from a substantially straight configuration (also referred to as “delivery configuration”) when disposed within a delivery sheath of a delivery apparatus (as described more fully below) to a helical configuration (also referred to as “deployed configuration,” as shown in FIG. 3) after being removed from the delivery sheath.

The coil 102 has a proximal end 102p and a distal end 102d. When being disposed within the delivery sheath (e.g., during delivery of the docking device into the vasculature of a patient), a body of the coil 102 between the proximal end 102p and distal end 102d can form a generally straight delivery configuration (e.g., without any coiled or looped portions) so as to maintain a small radial profile when moving through the patient's vasculature. After being removed from the delivery sheath and deployed at an implant position, the coil 102 can move from the delivery configuration to the helical deployed configuration and wrap around native tissue adjacent the implant position. For example, when implanting the docking device at the location of a native valve, the coil 102 can be configured to surround native leaflets of the native valve (and the chordae tendineae that connects native leaflets to adjacent papillary muscles, if present).

The docking device 100 can be releasably coupled to a delivery apparatus. For example, in certain examples, the docking device 100 can be coupled to a delivery apparatus via a release suture that can be configured to be tied to the docking device 100 and cut for removal (as described further below with reference to FIGS. 4 and 7). In one example, the release suture can be tied to the docking device 100 through an eyelet or eyehole 103 located adjacent to the proximal end 102p of the coil. In another example, the release suture can be tied around a circumferential recess that is located adjacent to the proximal end 102p of the coil 102.

In some examples, the docking device 100 in the deployed configuration can be configured to fit at the mitral valve position. In other examples, the docking device 100 can also be shaped and/or adapted for implantation at other native valve positions as well, such as at the tricuspid valve. In some examples, the geometry of the docking device 100 can be configured to engage the native anatomy, which can, for example, provide for increased stability and reduction of relative motion between the docking device 100, the prosthetic valve docked therein, and/or the native anatomy. Reduction of such relative motion can, among other things, prevent material degradation of components of the docking device 100 and/or the prosthetic valve docked therein and/or prevent damage or trauma to the native tissue.

As shown in FIG. 3, the coil 102 in the deployed configuration can include a leading turn 106 (or “leading coil”), a central region 108, and stabilization turn 110 (or “stabilization coil”). The central region 108 can possess one or more helical turns having substantially equal inner diameters. The leading turn 106 can extend from a distal end of the central region 108 and has a diameter greater than the diameter of the central region 108 (in one or more configurations). The stabilization turn 110 can extend from a proximal end of the central region 108 and has a diameter greater than the diameter of the central region 108 (in one or more configurations).

In certain examples, the central region 108 can include a plurality of helical turns, such as a proximal turn 108p in connection with the stabilization turn 110, a distal turn 108d in connection with the leading turn 106, and one or more intermediate turns 108m disposed between the proximal turn 108p and the distal turn 108d. In the example shown in FIG. 3, there is only one intermediate turn 108m between the proximal turn 108p and the distal turn 108d. In other examples, there are more than one intermediate turns 108m between the proximal turn 108p and the distal turn 108d. Some of the helical turns in the central region 108 can be full turns (i.e., rotating 360 degrees). In some examples, the proximal turn 108p and/or the distal turn 108d can be partial turns (e.g., rotating less than 360 degrees, such as 180 degrees, 270degrees, etc.).

The size of the docking device 100 can be generally selected based on the size of the desired prosthetic valve to be implanted into the patient. In certain examples, the central region 108 can be configured to retain a radially expandable prosthetic valve. For example, the inner diameter of the helical turns in the central region 108 can be configured to be smaller than an outer diameter of the prosthetic valve when the prosthetic valve is radially expanded so that additional radial tension can act between the central region 108 and the prosthetic valve to hold the prosthetic valve in place. The helical turns (e.g., 108p, 108m, 108d) in the central region 108 are also referred to herein as “functional turns.”

The stabilization turn 110 can be configured to help stabilize the docking device 100 in the desired position. For example, the radial dimension of the stabilization turn 110 can be significantly larger than the radial dimension of the coil in the central region 108, so that the stabilization turn 110 can flare or extend sufficiently outwardly so as to abut or push against the walls of the circulatory system, thereby improving the ability of the docking device 100 to stay in its desired position prior to the implantation of the prosthetic valve. In some examples, the diameter of stabilization turn 110 is desirably larger than the annulus, native valve plane, and atrium for better stabilization. In some examples, the stabilization turn 110 can be a full turn (i.e., rotating about 360 degrees). In some examples, the stabilization turn 110 can be a partial turn (e.g., rotating between about 180 degrees and about 270 degrees).

In one particular example, when implanting the docking device 100 at the native mitral valve location, the functional turns in the central region 108 can be disposed substantially in the left ventricle and the stabilization turn 110 can be disposed substantially in the left atrium. The stabilization turn 110 can be configured to provide one or more points or regions of contact between the docking device 100 and the left atrial wall, such as at least three points of contact in the left atrium or complete contact on the left atrial wall. In certain examples, the points of contact between the docking device 100 and the left atrial wall can form a plane that is approximately parallel to a plane of the native mitral valve.

As noted above, the leading turn 106 can have a larger radial dimension than the helical turns in the central region 108. The leading turn 106 can help more easily guide the coil 102 around and/or through the chordae tendineae geometry and adequately around all native leaflets of the native valve (e.g., the native mitral valve, tricuspid valve, etc.). For example, once the leading turn 106 is navigated around the desired native anatomy, the remaining coil (such as the functional turns) of the docking device 100 can also be guided around the same features. In some examples, the leading turn 106 can be a full turn (i.e., rotating about 360 degrees). In some examples, the leading turn 106 can be a partial turn (e.g., rotating between about 180 degrees and about 270 degrees). In some examples, when a prosthetic valve is radially expanded within the central region 108 of the coil, the functional turns in the central region 108 can be further radially expanded. As a result, the leading turn 106 can be pulled in the proximal direction and become a part of the functional turns in the central region 108.

As shown in FIGS. 3A-3D, in certain examples, at least a portion of the coil 102 can be surrounded by a first cover 112. In certain examples, the first cover 112 can cover an entire length of the coil 102. In certain examples, the first cover 112 covers only selected portion(s) of the coil 102. In certain examples, the first cover 112 can be coated on and/or bonded on the coil 102. In certain examples, the first cover 112 can be a cushioned, padded-type layer protecting the coil. The first cover 112 can be constructed of various native and/or synthetic materials. In one particular example, the first cover 112 can include expanded polytetrafluoroethylene (ePTFE). In certain examples, the first cover 112 can be fixedly attached to the coil 102 (e.g., by means of textured surface resistance, suture, glue, thermal bonding, or any other means) so that relative axial movement between the first cover 112 and the coil 102 is restricted or prohibited.

As shown in FIGS. 3A-3B, in some examples, the docking device 100 can also include a retention element 114 surrounding at least a portion of the coil 102 and at least being partially covered by the guard member 104. In some instances, the retention element 114 can comprise a braided material. In addition, the retention element 114 can provide a surface area that encourages or promotes tissue ingrowth and/or adherence, and/or reduce trauma to native tissue. For example, the retention element 114 can have a textured outer surface configured to promote tissue ingrowth. In certain instances, the retention element 114 can be impregnated with growth factors to stimulate or promote tissue ingrowth.

In certain examples, at least a portion of the first cover 112 can be surrounded by the retention element 114. In some examples, the first cover 112 can extend through an entire length of the retention element 114. In some examples, a distal end portion of the retention element 114 can extent axially beyond (i.e., positioned distal to) the distal end of the guard member 104, and a proximal end portion of the retention element 114 can extend axially beyond (i.e., positioned proximal to) a proximal end 105 of the guard member 104 to aid retention of prosthetic valve and tissue ingrowth.

The retention element 114 can be designed to interact with the guard member 104 to limit or resist motion of the guard member 104 relative to the coil 102. For example, the proximal end 105 of the guard member 104 can have an inner diameter that is about the same as an outer diameter of the retention element 114. As such, an inner surface of the guard member 104 at the proximal end 105 can frictionally interact or engage with the retention element 114 so that axial movement of the proximal end 105 of the guard member 104 relative to the coil 102 can be impeded by a frictional force exerted by the retention element 114.

As depicted in FIGS. 3A-3D, in certain examples, the guard member 104 can include an expandable member 116 and a second cover 118 surrounding an outer surface of the expandable member 116. In certain examples, the expandable member 116 surrounds at least a portion of the first cover 112. In certain examples, the first cover 112 can extend (completely or partially) through the expandable member 116.

In certain examples, the expandable member 116 can include a braided structure, such as a braided wire mesh or lattice. In certain examples, the expandable member 116 can include a shape memory material that is shape set and/or pre-configured to expand to a particular shape and/or size when unconstrained (e.g., when deployed at a native valve location). For example, the expandable member 116 can have a braided structure containing a shape memory alloy with superelastic properties, such as Nitinol. In certain examples, the expandable member 116 can have a braided structure containing a ternary shape memory alloy with superelastic properties, such as NiTiX where X can be chromium (Cr), cobalt (Co), zirconium (Zr), hafnium (Hf), etc. In certain examples, the expandable member 116 can comprise a metallic material that does not have the shape memory properties. Examples of such metallic material include cobalt-chromium, stainless steel, etc. In one specific example, the expandable member 116 can comprise nick-free austenitic stainless steel in which nickel can be completely replaced by nitrogen. In another specific example, the expandable member 116 can comprise cobalt-chromium or cobalt-nickel-chromium-molybdenum alloy with significantly low density of titanium. The number of wires (or fibers, strands, or the like) forming the braided structure can be selected to achieve a desired elasticity and/or strength of the expandable member 116. In certain examples, the number of wires used to braid the expanding member 116 can range from 16 to 128 (e.g., 32 wires, 48 wires, 64 wires, 96 wires, etc.). In certain examples, the braid density can range from 20 picks per inch (PPI) to 70 PPI, or from 25 PPI to 65 PPI. In one specific example, the braid density is about 36 PPI. In another specific example, the braid density is about 40 PPI. In certain examples, the diameter of the wires can range from about 0.002 inch to about 0.004 inch. In one particularly example, the diameter of the wires can be about 0.003 inch. In another example, the expandable member 116 can be a combination of braided wire (which can include a shape memory material or non-shape memory material) and a polymeric material and/or textile (e.g., polyethylene terephthalate (PET), polytetrafluoroethylene (PTFE), polyether ether ketone (PEEK), thermoplastic polyurethane (TPU), etc.). For example, the expandable member 116 can include a braided wireframe embedded in a polymeric material.

In some examples, the expandable member 116 can include a braided metallic wireframe coated with an elastomer (e.g., ePTFE, TPU, or the like), which can elastically deform as the braided wireframe expands and/or compresses. In some examples, the expandable member 116 can comprise a braid and/or weave that includes one or more metallic wires and one or more polymeric fibers. In other words, the metallic wires and the polymeric fibers can be interwoven together to define a braided structure. In some instances, the polymeric fibers can have the same or about the same diameter as the metallic wires. In other instances, the polymeric fibers can have a smaller diameter (e.g., microfibers) than the metallic wires, or vice versa.

In yet another example, the expandable member 116 can include a polymeric material, such as a thermoplastic material (e.g., PET, polyether ether ketone (PEEK), thermoplastic polyurethane (TPU), etc.), without a braided wireframe.

In certain examples, the expandable member 116 can include a foam structure. For example, the expandable member can include an expandable memory foam which can expand to a specific shape or specific pre-set shape upon removal of a crimping pressure (e.g., removal of the docking device 100 from the delivery sheath) prior to delivery of the docking device.

The expandable member 116 can extend radially outwardly from the coil 102 and is movable between a radially compressed (and axially elongated) state and a radially expanded (and axially foreshortened) state. That is, the expandable member 116 can axially foreshorten when it moves from the radially compressed state to the radially expanded state and can axially elongate when it moves from the radially expanded state to the radially compressed state.

The second cover 118 can be configured to be so elastic that when the expandable member 116 moves from the radially compressed (and axially elongated) state to the radially expanded (and axially foreshortened) state, the second cover 118 can also radially expand and axially foreshorten together with the expandable member 116. In other words, the guard member 104, as a whole, can move from a radially compressed (and axially elongated) state to a radially expanded (and axially foreshortened) state.

In certain examples, the second cover 118 can be configured to be atraumatic to native tissue and/or promote tissue ingrowth into the second cover 118. For example, the second cover 118 can have pores to encourage tissue ingrowth. In another example, the second cover 118 can be impregnated with growth factors to stimulate or promote tissue ingrowth. The second cover 118 can be constructed of any suitable material, including foam, cloth, fabric, and/or polymer, which is flexible to allow for compression and expansion of the second cover. In one example, the second cover 118 can include a fabric layer constructed from a thermoplastic polymer material, such as polyethylene terephthalate (PET).

The guard member 104 can constitute a part of a cover assembly for the docking device 100. In some examples, the cover assembly can also include the first cover 112. In some examples, the cover assembly can further include the retention element 114.

In certain examples, when the docking device 100 is in the deployed configuration, the guard member 104 can be configured to cover a portion of the stabilization turn 110 of the coil 102. In certain examples, the guard member 104 can be configured to cover at least a portion of the central region 108 of the coil 102, such as a portion of the proximal turn 108p. In certain examples, the guard member 104 can extend over the entirety of the coil 102.

In some examples, the guard member 104 can radially expand so as to help prevent and/or reduce paravalvular leakage. Specifically, the guard member 104 can be configured to radially expand such that an improved seal is formed closer to and/or against the prosthetic valve deployed within the docking device 100. In some examples, the guard member 104 can be configured to prevent and/or inhibit leakage at the location where the docking device 100 crosses between leaflets of the native valve (e.g., at the commissures of the native leaflets). For example, without the guard member 104, the docking device 100 may push the native leaflets apart at the point of crossing the native leaflets and allow for leakage at that point (e.g., along the docking device or to its sides). However, the guard member 104 can be configured to expand to cover and/or fill any opening at that point and inhibit leakage along the docking device 100.

In certain examples, when the docking device 100 is deployed at a native atrioventricular valve, the guard member 104 can cover predominantly a portion of the stabilization turn 110 and/or a portion of the central region 108. As such, the guard member 104 can help covering an atrial side of the atrioventricular valve to prevent and/or inhibit blood from leaking through the native leaflets, commissures, and/or around an outside of the prosthetic valve by blocking blood in the atrium from flowing in an atrial to ventricular direction (i.e., antegrade blood flow)—other than through the prosthetic valve. Positioning the guard member 104 on the atrial side of the valve can additionally or alternatively help reduce blood in the ventricle from flowing in a ventricular to atrial direction (i.e., retrograde blood flow).

In some examples, the guard member 104 can be positioned on a ventricular side of an atrioventricular valve to prevent and/or inhibit blood from leaking through the native leaflets, commissures, and/or around an outside of the prosthetic valve by blocking blood in the ventricle from flowing in a ventricular to atrial direction (i.e., retrograde blood flow). Positioning the guard member 104 on the ventricular side of the valve can additionally or alternatively help reduce blood in the atrium from flowing in the atrial direction to ventricular direction (i.e., antegrade blood flow)-other than through the prosthetic valve.

In some examples, a distal end portion 104d of the guard member 104 can be fixedly coupled to the coil 102 (e.g., via a suture), and a proximal end portion 104p of the guard member 104 can be axially moveable relative to the coil 102.

When the docking device 100 is retained within the delivery sheath in the substantially straight configuration, the expandable member 116 can be radially compressed by the delivery sheath and remain in the radially compressed (and axially elongated) state. The radially compressed (and axially elongated) expandable member 116 can contact the retention element 114 (see, e.g., FIG. 3B) or the first cover 112 (see, e.g., FIG. 3D) so that no gap or cavity exists between the retention element 114 and the expandable member 116 or between the first cover 112 (and/or the coil 102) and the expandable member 116.

After the docking device 100 is removed from the delivery sheath and changes from the delivery configuration to the deployed configuration, the guard member 104 can also move from a delivery configuration to a deployed configuration. In certain examples, a dock sleeve can be configured to cover and retain the docking device 100 within the delivery sheath when navigating the delivery sheath through the patient's native valve. The dock sleeve can also, for example, help to guide the docking device 100 around the native leaflets and chordae. Retraction of the dock sleeve relative to the docking device 100 can expose the guard member 104 and cause it to move from the delivery configuration to the deployed configuration. Specifically, without the constraint of the delivery sheath and the dock sleeve, the expandable member 116 can radially expand (and axially foreshorten) so that a gap or cavity 111 can be created between the retention element 114 and the expandable member 116 (see, e.g., FIG. 3A) and/or between the first cover 112 and the expandable member 116 (see, e.g., FIG. 3C).

Because the distal end portion 104d of the guard member 104 is fixedly coupled to the coil 102 and the proximal end portion 104p of the guard member 104 can be axially moveable relative to the coil 102, the proximal end portion 104p of the guard member 104 can slide axially over the first cover 112 and toward the distal end 102d of the coil 102 when the expandable member 116 moves from the radially compressed state to the radially expanded state. As a result, the proximal end portion 104p of the guard member 104 can be disposed closer to the proximal end 102p of the coil 102 when the expandable member 116 is in the radially compressed state than in the radially expanded state.

In certain examples, the second cover 118 can be configured to engage with the prosthetic valve deployed within the docking device 100 so as to form a seal and reduce paravalvular leakage between the prosthetic valve and the docking device 100 when the expandable member 116 is in the radially expanded state. The second cover 118 can also be configured to engage with the native tissue (e.g., the native annulus and/or native leaflets) to reduce paravalvular leakage between the docking device and/or the prosthetic valve and the native tissue.

In certain examples, when the guard member 104 is in the radially expanded state, the proximal end portion 104p of the guard member 104 can have a tapered shape, such that the diameter of the proximal end portion 104p gradually increases from the proximal end 105 of the guard member 104 to a distally located body portion of the guard member 104. This can, for example, help to facilitate loading the docking device into a delivery sheath of the delivery apparatus and/or retrieval and/or re-positioning of the docking device into the delivery apparatus during an implantation procedure. In addition, due to its small diameter, the proximal end 105 of the guard member 104 can frictionally engage with the retention element 114 so that the retention element 114 can reduce or prevent axial movement of the proximal end portion 104p of the guard member 104 relative to the coil 102.

Additional examples of the docking device and the guard member, as well as methods of assembling the docking device (e.g., attaching the guard member to the coil) are described in International Patent Application PCT/US2021/056150.

### Exemplary Delivery Apparatus

FIG. 4 illustrates an example delivery apparatuses 220 configured to deliver a docking device (such as docking device 100 described above with reference to FIG. 3) to a target implantation site (e.g., a heart and/or native valve of an animal, human, cadaver, and/or the like). In some examples, the delivery apparatus 220 can be a transcatheter delivery apparatus that can be used to guide the delivery of a docking device through a patient's vasculature, as explained above with reference to FIGS. 1 and 2A.

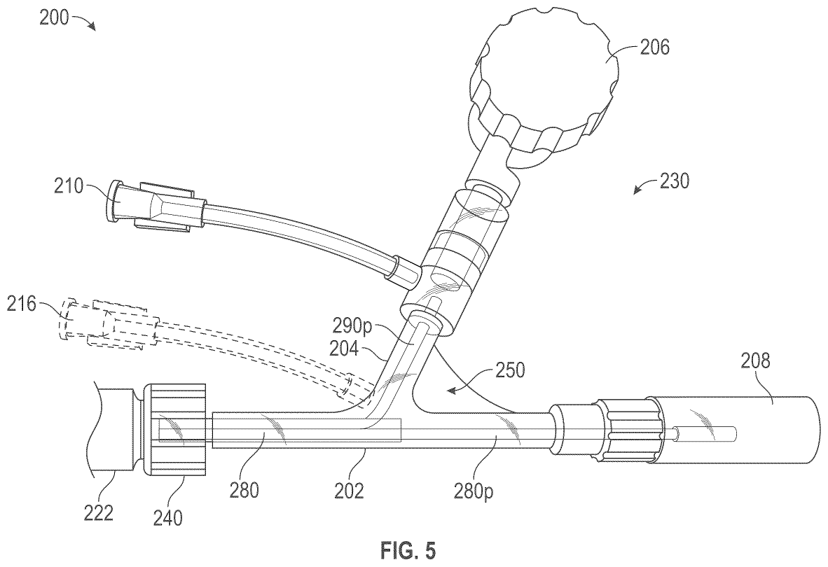

The exemplary delivery apparatus 220 is shown in FIG. 4 with a docking device 232 at least partially deployed from a distal end of the delivery apparatus 220 (e.g., for illustration purposes). In some examples, the docking device 232 can be the docking device 100 described above with reference to FIG. 3. The delivery apparatus 220 can include a handle assembly 200 and an outer shaft 260 (also referred to herein as “delivery sheath”) extending distally from the handle assembly 200. The handle assembly 200 can include a handle 222 and a hub assembly 230 extending from a proximal end of the handle 222. The outer shaft 260 extends distally from the handle 222 while the hub assembly 230 extends proximally from the handle 222. A more detailed view of the hub assembly 230, according to one example, is shown in FIG. 5, as described further below.

As shown in FIG. 4, the handle assembly 200 can include a handle 222 including one or more knobs, buttons, wheels, or the like. For example, the handle 222 can include knobs 224 and 226 which can be configured to control flexing of the delivery apparatus such as the outer shaft 260 and/or a sleeve shaft 280 described below. For example, by rotating a knob (e.g., 224 or 226), a curvature of the outer shaft 260 can be adjusted so that a distal end portion of the outer shaft 260 can be oriented in a desired angle.

As depicted in FIG. 7, the delivery apparatus 220 can include a pusher shaft 290 and a sleeve shaft 280 which are coaxially located within the outer shaft 260 and each have portions that extend into the handle assembly 200. In addition, the outer shaft 260 can be configured to be axially movable relative to the sleeve shaft 280 and the pusher shaft 290. In some examples, the pusher shaft 290 and the sleeve shaft 280 can respectively be the pusher shaft 32 and the sleeve shaft 34 described above with reference to FIG. 1. The pusher shaft 290 can be configured to deploy the docking device 232 from inside a distal end portion of the outer shaft 260, upon reaching the target implantation site. The sleeve shaft 280 can have a distal section (also referred to as “dock sleeve”) configured to cover the docking device 232 while inside the delivery apparatus 220 and while being positioned at the target implantation site. In some examples, the sleeve shaft 280 can have a lubricous and/or low-friction outer surface, e.g., by means of hydrophilic coating, at least at the dock sleeve. Such lubricous outer surface can improve the case of encircling the native anatomy and reduce risk of damage to the native tissue.

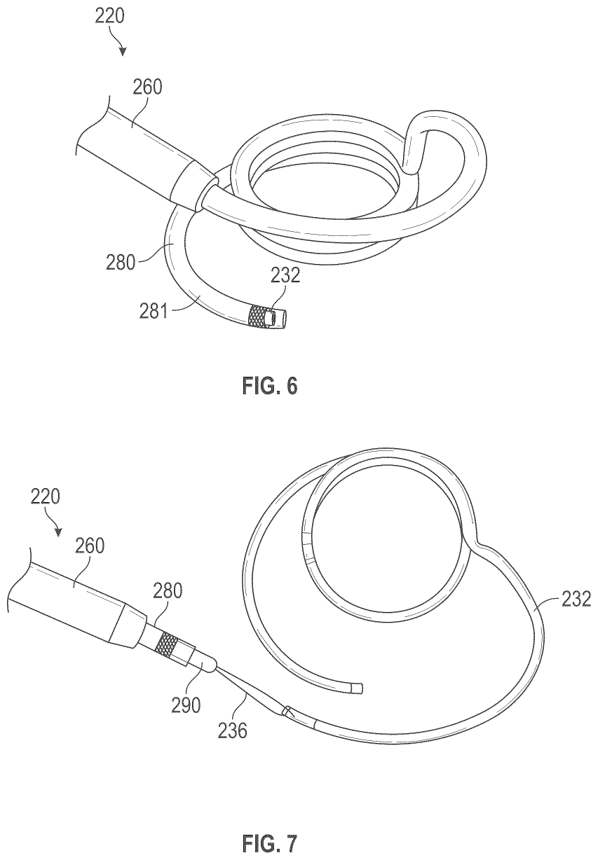

Further, the delivery apparatus 220 can be configured to adjust an axial position of the sleeve shaft 280 to remove the dock sleeve of the sleeve shaft 280 from the docking device 232, after implantation at the target implantation site. FIGS. 6-7 are perspective views showing the exemplary docking device 232 deployed from the outer shaft 260 of the delivery apparatus 220, covered by a dock sleeve 281 of the sleeve shaft 280 (FIG. 6), and the exemplary docking device 232 after the sleeve shaft 280 has been retracted back into the outer shaft 260 (FIG. 7).

In certain examples, deploying the docking device 232 from the outer shaft 260 can be accomplished by manipulating the pusher shaft 290 and sleeve shaft 280 using the hub assembly 230, as described further below. For example, by pushing the pusher shaft 290 in the distal direction while holding the outer shaft 260 in place or retracting the outer shaft 260 in the proximal direction while holding the pusher shaft 290 in place, or pushing the pusher shaft 290 in the distal direction while simultaneously retracting the outer shaft 260 in the proximal direction, the docking device 232 can be pushed out of a distal end of the outer shaft 260, thus changing from the delivery configuration to the deployed configuration.

In certain examples, the pusher shaft 290 and the sleeve shaft 280 can be actuated independently of each other. In certain examples, when deploying the docking device 232 from the outer shaft 260, the pusher shaft 290 and the sleeve shaft 280 can be configured to move together, in the axial direction, with the docking device 232. For example, actuation of the pusher shaft 290, to push against the docking device 232 and move it out of the outer shaft 260 can also cause the sleeve shaft 280 to move along with the pusher shaft 290 and the docking device 232. As such, the docking device 232 can remain being covered by the dock sleeve 281 of the sleeve shaft 280 during the procedure of pushing the docking device 232 into position at the target implantation site via the pusher shaft 290. Thus, when the docking device 232 is initially deployed at the target implantation site, the lubricous dock sleeve 281 can facilitate the covered docking device 232 to encircle the native anatomy.

As shown in FIGS. 4 and 7, during delivery, the docking device 232 can be coupled to the delivery apparatus 220 via a release suture 236 (or other retrieval line comprising a string, yarn, or other material that can be configured to be tied around the docking device and cut for removal) that can extend through the pusher shaft 290. As explained further below with reference to FIG. 5, the release suture 236 can extend through the delivery apparatus 220, through an inner lumen of the pusher shaft 290, to a suture lock assembly 206 (or “suture lock”) of the delivery apparatus 220. The suture lock assembly 206 can be used as the suture lock 40 depicted in FIG. 1.

As shown in FIGS. 4 and 5, the hub assembly 230 can include the suture lock assembly 206 and a sleeve handle attached thereto. A first example of the sleeve handle 234 is shown in FIG. 4 and a second example of the sleeve handle 208 is shown in FIG. 5. The hub assembly 230 can be configured to control the pusher shaft 290 and the sleeve shaft 280 of the delivery apparatus 220, together (e.g., move them axially together), while the sleeve handle (e.g., the sleeve handle 234 in FIG. 4 or the sleeve handle 208 in FIG. 5) can control an axial position of the sleeve shaft 280 relative to the pusher shaft 290. In this way, operation of the various components of the handle assembly 200 can actuate and control operation of the components arranged within the outer shaft 260. In some examples, as shown in FIGS. 4 and 5, the hub assembly 230 can be coupled to the handle 222 via a connector 240.

FIG. 5 shows an example of the hub assembly 230 for the delivery apparatus 220 in more detail. As shown, the hub assembly 230 can include a Y-shaped connector 250 (also referred to as an “adaptor”) having a straight section 202 (e.g., straight conduit) and at least one branch 204 (e.g., branch conduit), although, in some examples, it can include more than one branch. In some examples, the suture lock assembly 206 can be attached to the branch 204 and the sleeve handle (e.g., sleeve actuating handle) 208 can be arranged at a proximal end of the straight section 202.

The hub assembly 230 can be adapted and configured to allow a proximal extension 290p of the pusher shaft 290 (or another, similar pusher shaft) to extend to the suture lock assembly 206 arranged at the end of the branch 204, while a cut portion 280p (which may also be referred to as a proximal portion) of the sleeve shaft 280 extends to the sleeve handle 208, arranged at the end of the straight section 202. With this configuration, a medical professional can execute the deployment of the docking device (e.g., docking device 232 of FIG. 4) by manipulating the position of the handle assembly 200 (e.g., moving it in the axial direction) and also execute retraction of the sleeve shaft 280 (off of and away from the implanted docking device) by pulling back, in the axial direction, on the sleeve handle 208.

In this way, the sleeve shaft 280 and pusher shaft 290 can be configured to work together such that they can be moved simultaneously together when deploying and positioning the docking device at the native valve (e.g., by moving the entire hub assembly 230 forward and/or backward, in the axial direction), but can also to move independently so the pusher shaft 290 can hold the docking device in position while the sleeve shaft 280 is retracted off of the docking device (e.g., by holding the hub assembly 230 in place relative to the outer shaft 260 of the delivery apparatus 220 and/or other parts of the delivery apparatus 220 and/or docking device while pulling proximally on the sleeve handle 208 to withdraw the sleeve shaft 280). As described herein, the sleeve shaft 280 and the pusher shaft 290 can be coaxial along some, all, or a majority of the delivery apparatus 220 to facilitate this cooperative interaction.

As shown in FIGS. 4-5, the handle assembly 200 can further include one or more flushing ports to supply flush fluid to one or more lumens arranged within the delivery apparatus 220 (e.g., annular lumens arranged between coaxial components of the delivery apparatus 220) in order to reduce potential thrombus formation. One example where the delivery apparatus 220 includes three flushing ports (e.g., flushing ports 210, 216, and 218) is shown in FIG. 4. In an alternative example, the delivery apparatus 220 may not include flushing port 216 (e.g., as illustrated in FIG. 5).

Further details on delivery systems and apparatuses, such as delivery apparatus 220, that are configured to deliver a docking device to a target implantation site can be found in U.S. Patent Publication Nos. US2018/0318079, US2018/0263764, and US2018/0177594, which are all incorporated by reference herein in their entireties.

Additional details on a suture lock assembly and a pusher shaft and sleeve shaft assembly for a delivery apparatus for a docking device are described in International Patent Applications No. PCT/US2020/36577 and PCT/US2021/056150.

Additional details on a handle assembly of a delivery apparatus having one or more flushing ports configured to supply flush fluid to one or more lumens arranged within the delivery apparatus are described in International Patent Applications No. PCT/US2020/36577and PCT/US2021/059075.

### Exemplary Suture Lock Configured to Be Screwed onto an Adaptor

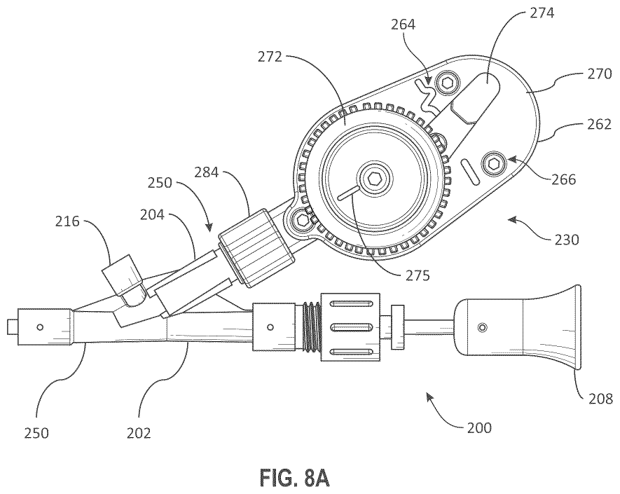

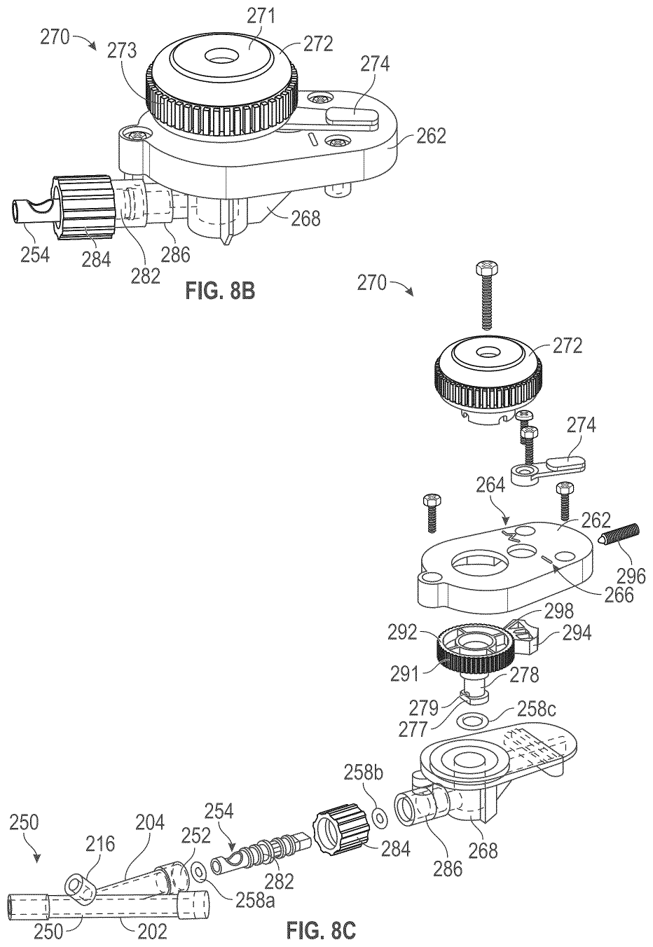

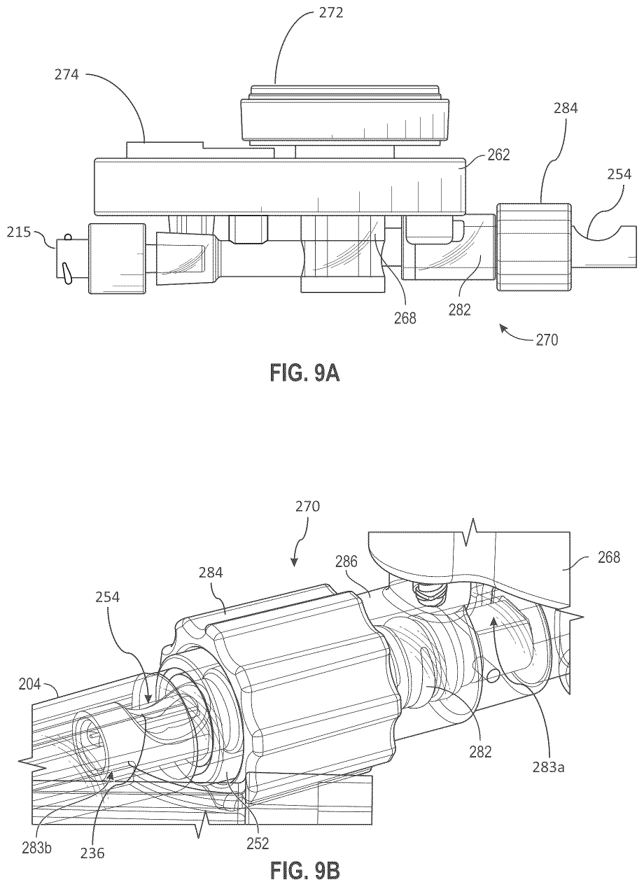

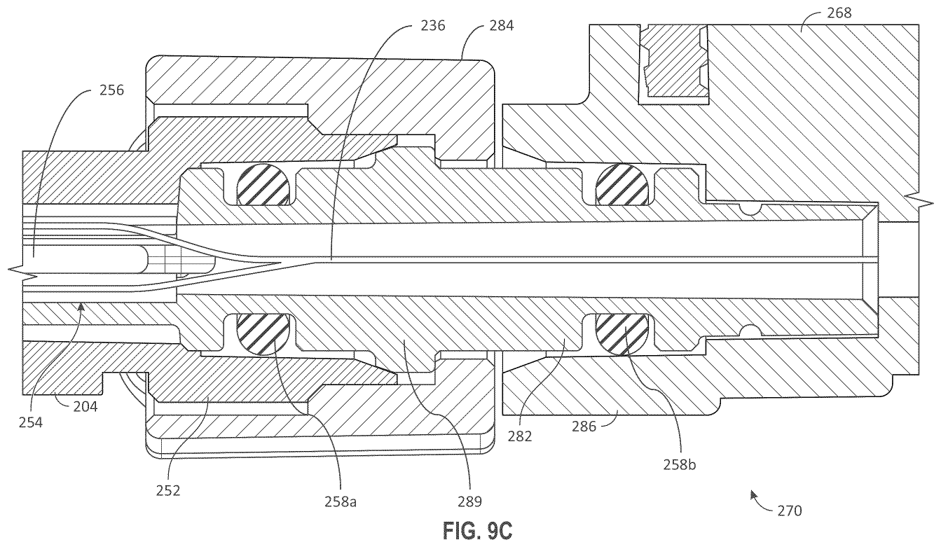

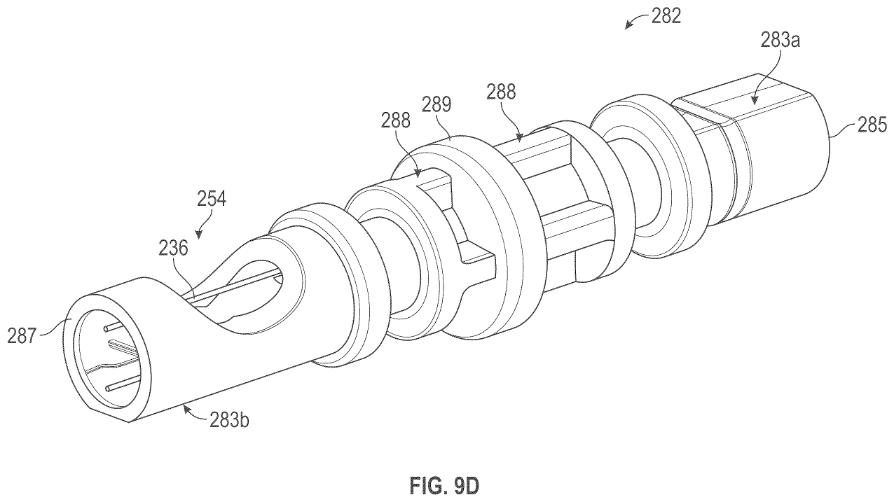

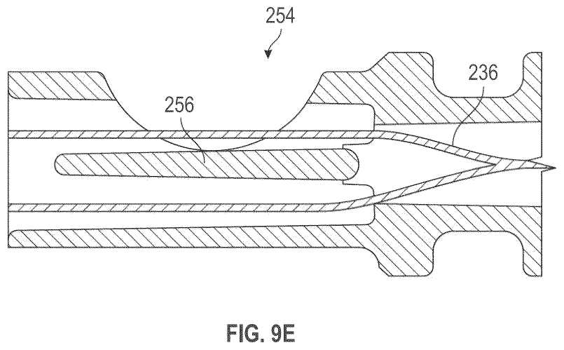

As shown in FIGS. 4-5 and introduced above, the delivery apparatus 220 can include a suture lock assembly 206 located on the branch 204 of the hub assembly 230 of the handle assembly 200. FIGS. 8A-9E show examples of a ratcheting suture lock 270 which may be used as the suture lock assembly 206 of the delivery apparatus 220 of FIGS. 4-5. As described below, the example suture lock 270 includes a release knob 284 that can be screwed onto an end (e.g., 252) of the adaptor 250.

As with the system illustrated in FIGS. 4-5, a proximal extension (e.g., 290p) of a pusher shaft (e.g., 290) can extend to the suture lock 270 at the end of branch 204, while a sleeve shaft (e.g., 280) can extend to a sleeve handle 208 at the end of the straight section 202. As depicted in FIG. 8A, the hub assembly 230 can include a flushing port 216 configured to allow flushing of one or more lumens within the delivery apparatus 220 to sterilize and/or maintain hemostasis within the delivery apparatus 220.

As with the system illustrated in FIGS. 4-5, a medical professional can deploy the docking device (e.g., 232) by manipulating the position of the handle assembly 200 and only adds one additional step to retract the sleeve by pulling back on the sleeve handle 208. The sleeve shaft (e.g., 280) and the pusher shaft (e.g., 290) can be configured to work together such that they can be moved simultaneously together when deploying and positioning the docking device at the native valve (e.g., by moving the entire hub assembly 230 and/or the Y-shaped connector 250 forward and/or backward). Additionally, the sleeve shaft and the pusher shaft can also be configured to move independently so that the pusher shaft can hold the docking device in position while the sleeve shaft is retracted off from the docking device (e.g., by holding the hub assembly 230 and/or the connector 250 in place relative to the outer shaft 260 and/or other parts of the delivery apparatus 220 and/or the docking device while pulling proximally on the sleeve handle 208 to withdraw the sleeve shaft). The sleeve shaft and the pusher shaft can be coaxial along some, all, or a majority of the delivery apparatus 220 to facilitate this working together, as explained above.

As shown in FIGS. 8A-9B, the suture lock 270 can include a rotator 272 (also may be referred to as a “rotatable handle”) to increase and decrease tension on a release suture 236 (shown in FIGS. 9B-9D) which can extend from the suture lock 270, through the branch 204, and through the handle 222 and the outer shaft 260 to connect to the docking device, as described above with reference to FIGS. 4 and 7.

In certain examples, the release suture 236 can be wrapped around a spool 278 of the suture lock 270 (see, e.g., FIG. 8C). The rotator 272 can be coupled to the spool 278, such that rotating the rotator 272 in a given direction can adjust (e.g., increase or decrease) tension on the release suture 236 traversing the delivery apparatus 220. Providing tension or slack to the release suture 236 via rotating the rotator 272 (and thus the spool 278) can bring the docking device 232 closer to or further away from the delivery apparatus 220, respectively.

As shown in FIG. 8B, in some examples, the rotator 272 can include one or more gripping portions or grips that increase an ease of gripping the rotator 272 (e.g., via a user's hand), without slipping. For example, the rotator 272 can include a first gripping portion 273 arranged around a circumference of the rotator 272 and that is configured to be gripped by a user during turning of the rotator 272. In some examples, the first gripping portion 273 can include a plurality of ridges to increase traction and ease of gripping. The rotator 272 can further include a second gripping portion 271 arranged on a top surface of the rotator 272. Further, in some examples, the first gripping portion 273 and/or the second gripping portion 271 can comprise a material having a lower durometer (e.g., reduced hardness).

In some examples, the suture lock 270 can further include a directional control mechanism which may include a directional selector 274 (e.g., in a form of a switch, as shown in FIGS. 8A-8C) that allows a medical practitioner or other user to select whether to increase or decrease slack in the release suture 236 traversing the delivery apparatus 220. For example, the directional selector 274 can be configured to allow a medical practitioner or other user to select a direction (e.g., increase or decrease tension), which will allow the rotator 272 to rotate in only one direction to prevent rotation in an incorrect direction.

For example, as shown in FIG. 8C, the spool 278 can include a gear 292 that can engage with a pawl 294 that allows rotation of the gear 292, and thus the rotator 272 and the spool 278, in only one direction. The direction the rotator 272 can be rotated depends on the orientation of the pawl 294, which is controlled by the directional selector 274. In some examples, as shown in FIGS. 8A and 8C, a top housing 262 can include a first icon 264 indicating a slack position of the directional selector 274 and a second icon 266 indicating a tension position of the directional selector 274.

In some examples, the directional control mechanism can be a ratcheting mechanism that limits directional movement from the rotator 272 by a medical practitioner or other user. As shown in FIGS. 8B and 8C, the gear 292 can be attached to the rotator 272, while the pawl 294 can be attached to the directional selector 274. The pawl 294 can be designed to engage with teeth 291 of the gear 292 such that the gear 292 can only rotate in one direction at a time. When the pawl 294 is actuated (e.g., pivoted) to a position (e.g., tension or slack), a spring plunger 296 can engage a back of the pawl 294, thereby retaining the pawl 294 in the selected direction/position (as shown in FIG. 8C). When engaged in one direction, one or more teeth 298 of the pawl 294 can interact with the teeth 291 on the gear 292. Additionally, a stop can be created to prevent the pawl 294 from moving bidirectionally, thus allowing the gear 292 to only move in one direction. The stop can be constructed in a number of ways including by making it a part of the top housing 262 or by adding additional materials (e.g., pins, spacers, etc.) inside the top housing 262 to prevent bidirectional movement of the pawl 294.

Additional details on the directional control mechanism for a suture lock (such as 270) are described in International Patent Applications No. PCT/US2020/36577.

In certain examples, the suture lock 270 can include a connector or connecting portion to attach the suture lock 270 to a handle assembly (e.g., 200). For example, the suture lock 270 can include a release bar 282 which extends into and couples with a bottom housing 268 of the suture lock 270 (see, e.g., FIGS. 8B-8C). In some examples, the release bar 282 can be bonded to the bottom housing 268 (e.g., via an adhesive, weld, or other non-removable fixing means). As shown in FIGS. 8B and 9A-9C, a release knob 284 can be arranged around a portion of the release bar 282, adjacent to a connecting portion 286 of the bottom housing 268. The release knob 284 can be configured to connect the suture lock 270 to the adaptor 250 of the delivery apparatus. As described above and depicted in FIG. 8A, the adaptor 250 can include a branch 204 and a straight section 202. In the depicted example, the release knob 284 can screw onto an end 252 of the adaptor 250 to secure the suture lock 270 to the adaptor 250. In some examples, the shape, size, and/or configuration of the adaptor 250 may be different than shown in FIG. 8A and may change based on the delivery apparatus to which the suture lock 270 is configured to be attached to (and used with).

In certain examples, when teeth of the release knob 284 engage both the end 252 of the adaptor 250 (or another adaptor of a delivery apparatus) and the release bar 282, the suture lock 270 can be coupled to the delivery apparatus and a suture cutting section 254 can be covered by the adaptor 250 (see, e.g., FIGS. 8B-8C and 9A-9E). In some examples, once the docking device (or other implant) is positioned in a desired position for release from the delivery apparatus, the release knob 284 can be unscrewed toward the bottom housing 268, and the suture lock 270 can be pulled proximally away from the adaptor 250 to expose the suture cutting section 254. In alternate examples, rotation of the release knob 284 toward the bottom housing 268 can expose the suture cutting section 254 without pulling the entire suture lock 270 away from the adaptor 250.

The suture cutting section 254 can be configured to allow for a user or medical practitioner to cut the release suture 236 that traverses the length of a delivery apparatus (e.g., as shown in FIGS. 4 and 7), to allow for the disconnection of a docking device from the delivery apparatus upon its deployment at a target implantation site.

In some examples, once the release suture 236 is wrapped around the docking device or implant (e.g., as shown in FIGS. 4 and 7) and routed through the delivery apparatus, through the release bar 282 (including across the suture cutting section 254, as shown in FIG. 9B), and into the bottom housing 268, the two suture ends of the release suture 236 can be threaded through two apertures 279 arranged in a bottom end of the spool 278 (see, e.g., FIG. 8C) and then tied to complete a suture loop. In some examples, the spool 278 can include a gap 277 in a flange at the bottom of the spool 278 that can prevent the release suture 236 from getting crushed during assembly of the top housing 262 and the bottom housing 268.

In some examples, as shown in FIG. 8A, the rotator 272 can include an indicator 275 to track a number of turns applied and locate the gap 277.

In some examples, as shown in FIGS. 9C and 9D, the release suture 236 can run longitudinally through the release bar 282 of the suture lock 270 and the two lines of the release suture 236 (forming the suture loop) split to cross a divider 256 arranged in the suture cutting section 254. The divider 256 can be configured to separate the two lines of release suture 236 such that only one line can be cut by a user or medical practitioner to release a docking device from the delivery apparatus. For example, the exposed portion of the release suture 236, as shown in FIG. 9D, can then be cut by a cutting mechanism, such as a blade, scissors, or the like. Once cut, the release suture 236 can be removed from the delivery apparatus, and the suture lock 270 can be attached back onto the adaptor 250 of the delivery apparatus, e.g., by screwing the release knob 284 onto the adaptor 250.

In certain examples, a seal can be included within the suture lock 270, e.g., by using a plurality of annular sealing elements (e.g., O-rings) 258a-c to prevent leakage of blood, saline, or other fluid through the system. For example, as shown in FIGS. 8C and 9C, the suture lock 270 can include a first, distal release bar O-ring 258a, a second, proximal release bar O-ring 258b, and a spool O-ring 258c. These O-rings 258a-c can be configured to seal the release suture path when the suture lock 270 is assembled, allowing for hemostasis when connected to a properly sealed delivery apparatus. The spool O-ring 258c can be configured to prevent leaks past the end of the release suture routing. The proximal release bar O-ring 258b can be configured to prevent leaks between the release bar 282 and the bottom housing 268. In some examples, this allows an adhesive or other bonding agent bonding the release bar 282 to the bottom housing 268 to act solely as a bond and does not require a sealing function. The distal release bar O-ring 258a can be configured to prevent leaks between the release bar 282 and the adaptor 250 while the release knob 284 is engaged. The release knob 284 can be designed such that the distal release bar O-Ring 258a seals the suture lock mechanism when there is any thread engagement with the adaptor 250 (e.g., there may be no variable sealing dependent on how tight the release knob is). In some examples, there may be a hole in the bottom housing 268 to act as a leak path in the event of a seal degradation.

As depicted in FIG. 9A, the suture lock 270 can include a flushing port 215 to allow flushing of one or more lumens within the delivery apparatus to reduce thrombus formation between components of the delivery apparatus, maintain hemostasis within a delivery apparatus, and/or to sterilize a delivery apparatus. In certain cases, the flushing port 215 can be configured to allow flushing lumens independently if a single flush line becomes clogged and/or is not maintaining hemostasis in a delivery apparatus. In certain cases, the flushing port 215 can be an open port to allow constant flow through a delivery apparatus. In certain examples, the flushing port 215 can be configured as self-sealing such that fluids can be introduced into a delivery apparatus as needed by a practitioner without requiring constant flow. The flushing port 215 depicted in FIG. 9A allows for an additional flush line to be connected akin to multiple flushing ports, such as the ones illustrated in FIG. 4 and discussed above.

In certain examples, the suture lock 270 can have segments that are keyed to prevent rotation of a suture lock 270 around a handle assembly, thus preventing twisting of suture lines and/or increasing case of access for a practitioner. Keying of the suture lock 270 can be accomplished various ways, such as by creating a specific non-round shape in the components, use of pins, grooves, or any other methodology to maintain a non-rotating fit between the suture lock 270 and an outer housings or handle assembly. For example, as shown in FIG. 9B, either end of the release bar 282 can be shaped to form first and second keyed connections 283a and 283b between the release bar 282 and the bottom housing 268 and between the release bar 282 and the adaptor 250, respectively. For example, a proximal end 285 of the release bar 282 can be shaped to form the first keyed connection 283a and a distal end 287 of the release bar 282 can be shaped to form the second keyed connection 283b.

In some examples, as shown in FIGS. 9D, the release bar 282 can include one or more supporting ribs 288 arranged on a center portion of the release bar 282, the center portion arranged between the distal end 287 and the proximal end 285 of the release bar 282. In certain examples, the supporting ribs 288 can include a plurality of axially-extending ribs 288 that are arranged around a circumference of the release bar 282, on either side of a central ring element 289 that extends around the circumference of the release bar 282.

### Exemplary Suture Lock Comprising a Lock and Release Mechanism

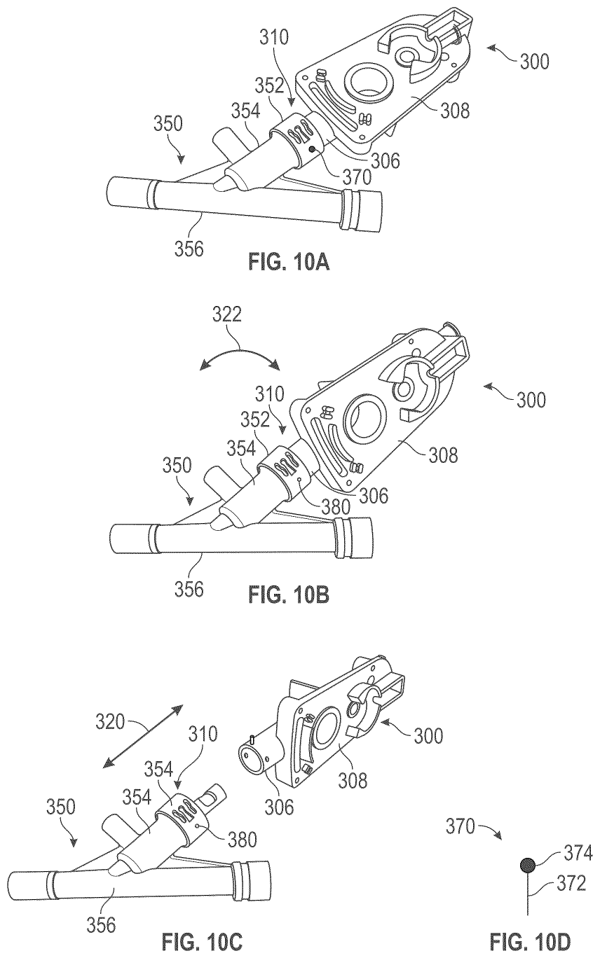

FIGS. 10A-10C show a suture lock 300 configured to be releasably connected to an adaptor 350 of a handle assembly (e.g., 200), according to another example. Specifically, FIG. 10A depicts the suture lock 300 being locked to the adaptor 350, FIG. 10B depicts the suture lock 300 being unlocked and partially rotated relative to the adaptor 350, and FIG. 10C depicts the suture lock 300 being disconnected from the adaptor 350. The suture lock 300 can be used as the suture lock assembly 206 of the delivery apparatus 220 of FIGS. 4-5. The adaptor 350 can be similar to the Y-shaped connector 250 described above. For example, the adaptor 350 can include a branch 354 and a straight section 356.

In the depicted example, the suture lock 300 can be releasably connected to the adaptor 350 via a lock and release mechanism 310, which is embodied in a connecting portion 306 of a bottom housing 308 (similar to the connecting portion 286 of the bottom housing 268) of the suture lock 300 and an end portion 352 of the branch 354 of the adaptor 350. In other examples, the lock and release mechanism 310 can be embodied in other parts of the suture lock 300 and the adaptor 350.

With the lock and release mechanism 310, the suture lock 300 can be securely locked to the adaptor 350 by an insertion step followed by a rotation step. For example, the connecting portion 306 of the suture lock 300 can be inserted into the end portion 352 of the adaptor 350, and then rotating the suture lock 300 relative to the adaptor 350 for a partial turn (e.g., a quarter turn, a half turn, etc.) can securely lock the suture lock 300 to the adaptor 350. Alternatively, the lock and release mechanism 310 can be configured to securely lock the suture lock 300 to the adaptor 350 by inserting the end portion 352 of the adaptor 350 into an inner lumen of the connecting portion 306 of the suture lock 300 and then rotating the suture lock 300 relative to the adaptor 350.

Conversely, with the lock and release mechanism 310, the suture lock 300 can be easily unlocked from the adaptor 350 by a rotation step followed by a pulling step. For example, to remove the suture lock 300 from the adaptor 350, a user can grab the suture lock 300, rotate it relative to the adaptor 350 for a partial turn (e.g., a quarter turn, a half turn, etc.), and then pull it off from the adaptor 350.

Thus, the steps involved in unlocking the suture lock 300 from the adaptor 350 are in reverse sequence and opposite to the steps involved in locking the suture lock 300 to the adaptor 350. For example, the insertion (for locking) and the pulling (for unlocking) are operated in opposite directions (as indicated by the double-sided straight arrow 320 in FIG. 10C) along a longitudinal axis, which is coaxial with the connecting portion 306 of the suture lock 300 and the end portion 352 of the adaptor 350. Likewise, the rotational directions for locking and unlocking are in opposite directions (as indicated by the double-sided curved arrow 322 in FIG. 10B) but the degree of rotation is about the same. For example, if the locking step involves a quarter turn in a clockwise direction when viewed from the suture lock 300, the unlocking step involves a quarter turn in a counterclockwise direction when viewed from the suture lock 300; or vice versa.

Optionally, when in the locked configuration, a safety feature can be added (in addition to the lock and release mechanism 310) to increase the strength of the joint between the suture lock 300 and the adaptor 350 and prevent unintentional unlocking. In certain examples, the safety feature can be a lock pin 370 (see, e.g., FIGS. 10A and 10D) configured to fit through a transverse hole 380 (see, e.g., FIGS. 10B-10C) extending through both the connecting portion 306 of the suture lock 300 and the end portion 352 of the adaptor 350. The transverse hole 380 can comprise a first hole on the connecting portion 306 of the suture lock 300 and a second hole on the end portion 352 of the adaptor 350. When the suture lock 300 is locked to the adaptor 350, the first hole can be configured to align with the second hole so as to form the transverse hole 380.

In certain examples, the lock pin 370 can include a pin portion 372 configured to be inserted into the transverse hole 380 and an enlarged head portion 374 having a larger diameter than the transverse hole 380. When the pin portion 372 is inserted into the transverse hole 380, the enlarged head portion 374 can be configured to stay outside the transverse hole 380 (see, e.g., FIG. 10A) and is easily grabbed by a user.

Inserting the lock pin 370 into the transverse hole 380 can enhance the locking (provided by the lock and release mechanism 310) between the suture lock 300 and the adaptor 350, and prevent unintentional rotation of the suture lock 300 relative to the adaptor 350. In order to unlock and remove the suture lock 300 from the adaptor 350, the user needs to intentionally remove the lock pin 370 from the transverse hole 380 (see, e.g., FIGS. 10B-10C).

As described herein, the suture lock 300 can be coupled to and/or released from the adaptor 350 without the need of operating a release knob (e.g., 284) described above. In certain circumstances, the lock and release mechanism 310 described herein may be more accessible than the release knob. In addition, the lock and release mechanism 310 may reduce the risk of air embolism, e.g., introduced by back-driving of the release knob in certain instances. Further, compared to the release knob (e.g., 284) which can be tightened to variable degrees, the lock and release mechanism 310 creates discreet locking and unlocking configurations.

Representative examples of the lock and release mechanism 310 are described more fully below.

### Exemplary Lock and Release Mechanisms Using Interference Fit

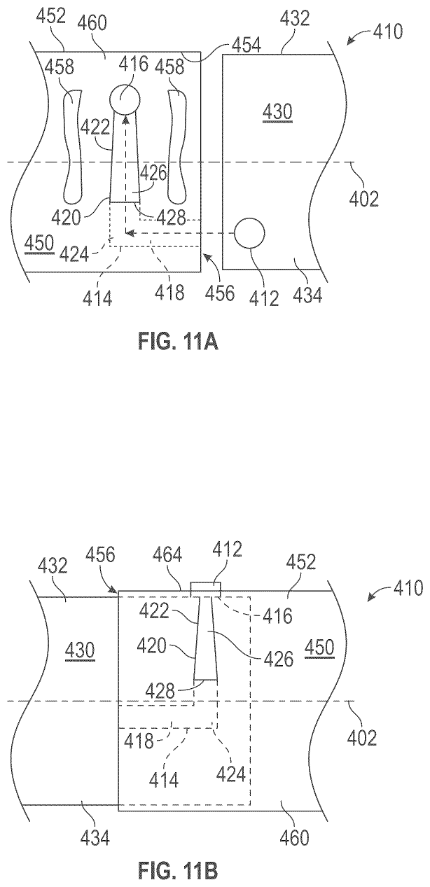

FIGS. 11A-11B depicts a lock and release mechanism 410, which can be one example of the lock and release mechanism 310 depicted in FIGS. 10A-10C, through which a suture lock (e.g., 300) can be connected to and/or disconnected from an adaptor (e.g., 350).

As described more fully below, the lock and release mechanism 410 is different from the traditional bayonet mechanism for releasably coupling two mating components, where a peg on one of the mating components can slide through an L-shaped slot and into a cutout area on another mating component. The traditional bayonet mechanism requires either a tension force (e.g., exerted by a compression spring, a gasket, etc.) or a compression force (e.g., exerted by a tension spring, a taut suture, etc.) created between the slot and the peg to hold the peg in the cutout area and prevent rotational movement between the two mating components. For example, if the two components are held together in tension, they must be pushed against one another to overcome the tension force in order to enable the rotation. Conversely, if the two components are held together in compression, they must be pulled away from one another to overcome the compression force in order to enable the rotation. Although a release suture (e.g., 236) may also be used to create a compression force, the release suture cannot be relied upon to persistently create the compression used in a bayonet mechanism to lock/unlock between a suture lock and an adaptor. For example, while the release suture is tightened and taut during the initial procedure of delivering the prosthetic implant (e.g., the docking device 100 or 232) to a target implantation site, the release suture is cut loose and have slacks after the prosthetic implant is deployed. As a result, the release suture loses the compression force and the suture lock can be accidentally unlocked from the adaptor. As described below, the lock and release mechanism 410 does not rely on compression or tension between two mating parts to hold them in place. Instead, the lock and release mechanism 410 uses an interference fit mechanism to lock/unlock between the suture lock and the adaptor.

As shown in FIG. 11A, the lock and release mechanism 410 can include a peg 412 (which may also be referred to as a “pin”), a slot 414 (may also be referred to as a “track” or a “channel”), and a retaining portion 416 connected to the slot 414. The peg 412 is configured to slide or travel through the slot 414 and received/retained within the retaining portion 416. The size and shape of the retaining portion 416 can be configured to match those of the peg 412. In certain examples, the retaining portion 416 can be considered as an enlarged portion of the slot 414, and the other portion of the slot 414 can be referred to as a “track portion” of the slot 414.

As shown in FIG. 11A, the peg 412 is located on a first component 430 and the slot 414 is located on a second component 450. In certain examples, the first component 430 can be a suture lock (e.g., 300) and the second component 450 can be an adaptor (e.g., 350). In such case, the peg 412 can be located on a connecting portion (e.g., 306) of the suture lock and the slot 414 can be located on an end portion (e.g., 352) of the adaptor. In addition, the first component 430 can include a spool (e.g., 278) configured to connect to a release suture (e.g., 236) which extends through an inner bore (e.g., 456) of the second component 450 and is tied to the prosthetic implant (e.g., the docking device 100 or 232). In some examples, the first component 430 can be an adaptor (e.g., 350) and the second component 450 can be a suture lock (e.g., 300). In such case, the slot 414 can be located on a connection portion (e.g., 306) of the suture lock and the peg 412 can be located on an end portion (e.g., 352) of the adaptor. In addition, the second component 450 can include a spool (e.g., 278) configured to connect to a release suture (e.g., 236) which extends through a lumen of the first component 430 and is tied to the prosthetic implant (e.g., the docking device 100 or 232).

In the depicted example, the first component 430 includes a male connection portion 432 having a main body 434, and the peg 412 extends radially outwardly from the main body 434. In certain examples, the peg 412 can be formed as an integrated part of the main body 434. In certain examples, the peg 412 can be separately prepared and then attached to the main body 434, e.g., via thermal molding, adhesives, press fit, or the like. The second component 450 includes a female connection portion 452 configured to receive the male connection portion 432 of the first component 430. The female connection portion 452 has a wall 460. An inner surface 454 of the wall 460 defines an inner bore 456. The slot 414 can extend along the inner surface 454. The retaining portion 416 can be connected to a terminal end of the slot 414 and sized and shaped to receive the peg 412. The slot 414 and/or the retaining portion 416 can be formed on the wall 460, e.g., via laser cutting, etching, or other means, or directly added to the female connection portion 452 as a molded feature, machined, etc.

The first component 430 and the second component 450 can be configured to be movable between a locked configuration and a release (or unlocked) configuration. When in the locked configuration, the peg 412 is positioned within the retaining portion 416 and the slot 414 is configured to resist rotational movement of male connection portion 432 relative to the female connection portion 452. When in the release configuration, the peg 412 is positioned within the slot 414.

In various examples, the first component 430 and the second component 450 can be moved to the locked configuration by inserting the main body 434 into the inner bore 456 and rotating the male connection portion 432 relative to the female connection portion 452 in a first direction for less than one revolution about an axial axis 402 of the main body 434. When the first component 430 is connected to the second component 450, the main body 434 and the inner bore 456 are coaxial (i.e., they have the common axis 402).

When in the locked configuration, the first component 430 and the second component 450 can be moved to the release configuration by rotating the male connection portion 432 relative to the female connection portion 452 in a second direction opposite to the first direction for less than one revolution about the axial axis 402 of the main body 434. Then the main body 434 can be pulled away from the inner bore 456.

The first direction described above can be clockwise or counterclockwise when viewed from the first component 430. As described herein, the less than one resolution rotation can correspond to rotation between 10 degrees and 350 degrees, or between 45 degrees and 315 degrees, or between 90 degrees and 180 degrees, inclusive. In one particular example, the rotation can be a quarter turn, e.g., about 90 degrees. In another particular example, the rotation can be an eight turn, e.g., about 45 degrees.

As shown in FIG. 11A, the slot 414 can include an axial portion 418 (may also be referred to as a “first slot portion” or “first track portion”) extending parallel to the axial axis 402 and a circumferential portion 420 (may also be referred to as a “second slot portion” or “second track portion”) that is connected to the axial portion 418 at a corner 424 and extends circumferentially about the axial axis 402.

In the depicted example, the retaining portion 416 is connected to a terminal end of the circumferential portion 420. As shown, the circumferential portion 420 can include a terminal portion 422 adjacent to the retaining portion 416. In certain examples, the circumferential portion 420 can have a tapered shape such that a diameter of the circumferential portion 420 can progressively decreases from the corner 424 to the terminal portion 422 and/or the retaining portion 416.

In certain examples, the retaining portion 416 can be a cutout extending radially through the wall 460 so that the peg 412 received in the retaining portion 416 can be exposed through the cutout, thus providing a visual confirmation the first component 430 and the second component 450 are in the locked configuration. In certain examples, the peg 412 received in the retaining portion 416 can extend radially outwardly beyond an outer surface 464 of the wall 460 (see, e.g., FIG. 11B) such that the wall 460 can prevent the peg 412 from accidentally slipping out of the retaining portion 416.

In certain examples, the circumferential portion 420 can include a cutout window 426 (e.g., formed by laser cutting or the like) extending radially through the wall 460 of the female connection portion 452 so as to expose the peg 412 when the peg 412 is moved to the cutout window 426. In certain examples, the cutout window 426 can include the terminal portion 422. For example, the cutout window 426 can extend from the retaining portion 416 to a cutout edge 428 that is spaced apart from the corner 424.

According to certain examples, the terminal portion 422 can be deflectable. Thus, the terminal portion 422 may also be referred to as a “deflectable portion.” When the peg 412 is not inserted into the terminal portion 422, the terminal portion 422 can have a smaller diameter than the peg 412. But when the peg 412 is received in the terminal portion 422, the terminal portion 422 can be dilated by the peg 412. Thus, the peg 412 can form an interference fit with the terminal portion 422, while an external force (e.g., by rotating the male connection portion 432 relative to the female connection portion 452) can push the peg 412 to slide through the terminal portion 422 of the slot 414.

To lock the first component 430 to the second component 450, the male connection portion 432 can be inserted into the female connection portion 452 so that the peg 412 can slide through the axial portion 418 of the slot 414. After reaching the corner 424, the male connection portion 432 can be rotated relative to the female connection portion 452 so that the peg 412 can slide through the circumferential portion 420 (the terminal portion 422 is dilated to accommodate the sliding peg 412) and into the retaining portion 416. Because the terminal portion 422 has a smaller diameter than the peg 412, once the peg 412 is received in the retaining portion 416, the terminal portion 422 will resist the peg 412 from moving out of the retaining portion 416. As a result, the male connection portion 432 will be inhibited from rotating relative to the female connection portion 452 (i.e., locked).

To unlock the first component 430 from the second component 450, the male connection portion 432 can be rotated relative to the female connection portion 452 (in opposite direction of the locking operation) so that a sufficiently large external force can urge the peg 412 out of the retaining portion 416 and into the circumferential portion 420 (i.e., unlocked), e.g., by dilating and overcoming the resistance exerted by the terminal portion 422.

In certain examples, the female connection portion 452 can include at least one relief channel 458 extending parallel to the circumferential portion 420 and configured to allow the terminal portion 422 to axially dilate when the peg 412 moves through the terminal portion 422. The relief channel 458 can be a cutout (e.g., formed by laser cutting or the like, and/or directly added as a molded features, machined, etc.) extending radially through the wall 460 of the female connection portion 452, and the void space in the relief channel 458 allows material around the terminal portion 422 to flex during insertion of the peg 412 into the terminal portion 422. In certain examples, a circumferential length of the relief channel 458 can be about the same as or larger than a circumferential length of the cutout window 426.

In the example depicted in FIG. 11A, two relief channels 458 about the same size and shape are symmetrically located on both sides of the terminal portion 422. In other examples, the two relief channels 458 can have different size and/or shapes, and can be located asymmetrically about the terminal portion 422. In still other examples, there can be only one relief channel or more than two relief channels 458 around the terminal portion 422.

In lieu of or in addition to the relief channel 458, the wall 460 of the female connection portion 452 can comprise a deformable material (e.g., rubber, copper, aluminum, nickel titanium alloy, magnesium alloy, etc.) around the terminal portion 422 such that when the peg 412 moves through the terminal portion 422, the peg 412 can compress the deformable material and axially dilate the terminal portion 422.

According to certain examples, the peg 412 can be self-expandable. For example, the peg 412 can be movable between a compressed state and an expanded state, and be configured to be biased toward the expanded state. For example, the peg 412 can remain in the expanded state when no external force is applied. The peg 412 can be moved to the compressed state when a squeezing force is applied to the peg 412 and return to the expanded state when the squeeze force is removed. The peg 412 can have a larger diameter in the expanded state than the compressed state. Various techniques can be used to design such a self-expandable peg 412, such as using self-expandable materials (e.g., rubber, nickel titanium alloy, etc.) and/or structural configurations (e.g., coil, spring, etc.).

In certain examples, the retaining portion 416 can be configured to retain the peg 412 in the expanded state and the slot 414 can be configured to receive the peg 412 in the compressed state. For example, the retaining portion 416 can have a diameter that matches the diameter of the peg 412 in the expanded state. In one example, the slot 414 can have a substantially constant diameter that matches the diameter of the peg 412 in the compressed state. In another example, the diameter of the slot 414 may vary (e.g., the diameter of the axial portion 418 can be larger than the diameter of the circumferential portion), and at least the terminal portion 422 can have a diameter that matches the diameter of the peg 412 in the compressed state. Thus, the peg 412 can be press fit into the terminal portion 422 of the slot 414, and an external force (e.g., by rotating the male connection portion 432 relative to the female connection portion 452) can push the peg 412 (in the compressed state) through the slot 414.

Similarly, to lock the first component 430 to the second component 450, the male connection portion 432 can be inserted into the female connection portion 452 so that the peg 412 can slide through the axial portion 418 of the slot 414. After reaching the corner 424, the male connection portion 432 can be rotated relative to the female connection portion 452 so that the peg 412 can slide through the circumferential portion 420 (the peg 412 is squeezed to the compressed state in the terminal portion 422) and into the retaining portion 416. Once the peg 412 is received in the retaining portion 416, the peg 412 can return to the expanded state. Thus, the narrower terminal portion 422 will resist the expanded peg 412 from moving out of the retaining portion 416. As a result, the male connection portion 432 will be inhibited from rotating relative to the female connection portion 452 (i.e., locked).

To unlock the first component 430 from the second component 450, the male connection portion 432 can be rotated relative to the female connection portion 452 (in opposite direction of the locking operation) so that a sufficiently large external force can urge the peg 412 out of the retaining portion 416 and into the circumferential portion 420 (i.e., unlocked), e.g., by compressing the peg 412 and press-fitting the peg 412 into the terminal portion 422.

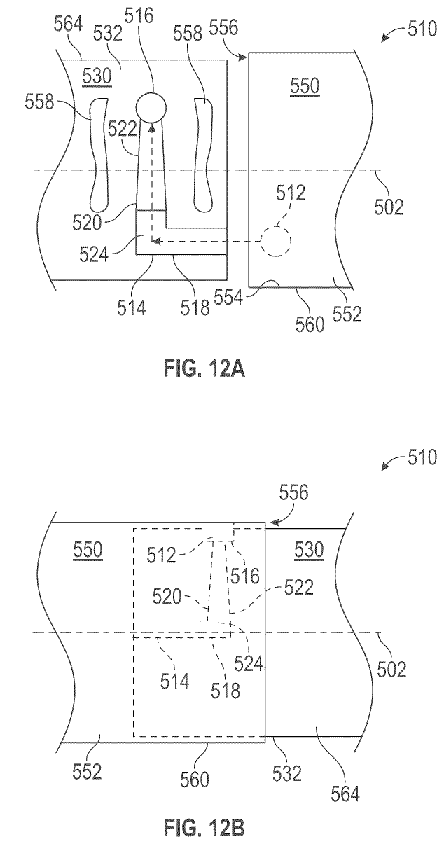

FIGS. 12A-12B depicts a lock and release mechanism 510, which can be another example of the lock and release mechanism 310 depicted in FIGS. 10A-10C, through which a suture lock (e.g., 300) can be connected to and/or disconnected from an adaptor (e.g., 350). Similar to 410, the lock and release mechanism 510 uses an interference fit mechanism to lock/unlock between the suture lock and the adaptor.

As shown in FIG. 12A, the lock and release mechanism 510 can include a peg 512, a slot 514, and a retaining portion 516 connected to the slot 514. The peg 512 is configured to slide or travel through the slot 514 and received/retained within the retaining portion 516. The size and shape of the retaining portion 516 can be configured to match those of the peg 512. In certain examples, the retaining portion 516 can be considered as an enlarged portion of the slot 514.

As shown in FIG. 12A, the peg 512 is located on a first component 550 and the slot 514 is located on a second component 530. In certain examples, the first component 550 can be a suture lock (e.g., 300) and the second component 530 can be an adaptor (e.g., 350). In such case, the peg 512 can be located on a connecting portion (e.g., 306) of the suture lock and the slot 514 can be located on an end portion (e.g., 352) of the adaptor. In addition, the first component 550 can include a spool (e.g., 278) configured to connect to a release suture (e.g., 236) which extends through a lumen of the second component 530 and is tied to the prosthetic implant (e.g., the docking device 100 or 232). In some examples, the first component 550 can be an adaptor (e.g., 350) and the second component 530 can be a suture lock (e.g., 300). In such case, the slot 514 can be located on a connection portion (e.g., 306) of the suture lock and the peg 512 can be located on an end portion (e.g., 352) of the adaptor. In addition, the second component 530 can include a spool (e.g., 278) configured to connect to a release suture (e.g., 236) which extends through an inner bore (e.g., 556) of the first component 550 and is tied to the prosthetic implant (e.g., the docking device 100 or 232).

In the depicted example, the first component 550 includes a female connection portion 552. The female connection portion 552 has a wall 560. An inner surface 554 of the wall 560 defines an inner bore 556. The peg 512 extends radially inwardly from the inner surface 554 (i.e., extending from the wall 560 into the inner bore 556). The second component 530 includes a male connection portion 532 configured to be inserted into the inner bore 556 of the female connection portion 552. The male connection portion 532 has an outer surface 564. The slot 514 can extend along the outer surface 564, and the retaining portion 516 can be connected to the slot 514 and configured to receive the peg 512. When connected, the male connection portion 532 and the female connection portion 552 can share a common axial axis 502.

Similarly, the first component 550 and the second component 530 can be configured to be movable between a locked configuration and a release (or unlocked) configuration. When in the locked configuration, the peg 512 is positioned within the retaining portion 516 and the slot 514 is configured to resist rotational movement of male connection portion 532 relative to the female connection portion 552. When in the release configuration, the peg 512 is positioned within the slot 514.

Unlike the lock and release mechanism 410 where the peg 412 extends radially outwardly from the male connection portion 432 and is received in the slot 414 and/or the retaining portion 416 located on the female connection portion 452, the lock and release mechanism 510 reverses the peg-slot configuration. That is, the peg 512 extends radially inwardly from the female connection portion 552 and is received in the slot 514 and/or the retaining portion 516 located on the male connection portion 532. Despite such difference, the lock and release mechanism 510 can lock/unlock between the first component 550 and the second component 530 in a similar manner as the lock and release mechanism 410 locks/unlocks between the first component 430 and the second component 450, as described above.

For example, the first component 550 and the second component 530 can be moved to the locked configuration by inserting the male connection portion 532 into the inner bore 556 and rotating the male connection portion 532 relative to the female connection portion 552 in a first direction for less than one revolution about the axial axis 502. Similarly, when in the locked configuration, the first component 550 and the second component 530 can be moved to the release configuration by rotating the male connection portion 532 relative to the female connection portion 552 in a second direction opposite to the first direction for less than one revolution about the axial axis 502.

The peg 512, the slot 514, and the retaining portion 516 can have the same shape and/or size (and be formed/created in the same way) as the peg 412, the slot 414, and the retaining portion 416, respectively. For example, the slot 514 can have an axial portion 518 extending parallel to the axial axis 502 and a circumferential portion 520 that is connected to the axial portion 518 at a corner 524 and extends circumferentially about the axial axis 502. The retaining portion 516 can be connected to a terminal end of the circumferential portion 520. The circumferential portion 520 can include a terminal portion 522 adjacent to the retaining portion 516. In certain examples, the circumferential portion 520 can have a tapered shape such that a diameter of the circumferential portion 520 can progressively decreases from the corner 524 to the terminal portion 522 and/or the retaining portion 516.

Similar to 422, the terminal portion 522 can be deflectable. When the peg 512 is not inserted into the terminal portion 522, the terminal portion 522 can have a smaller diameter than the peg 512. But when the peg 512 is received in the terminal portion 522, the terminal portion 522 can be dilated by the peg 512. Thus, the peg 512 can form an interference fit with the terminal portion 522, while an external force (e.g., by rotating the male connection portion 532 relative to the female connection portion 552) can push the peg 512 to slide through the terminal portion 522 of the slot 514. Likewise, the deflectable terminal portion 522 can be achieved by having one or more relief channels 558 (similar to 458) around the circumferential portion 520, and/or using a deformable material around the terminal portion 522.

Alternatively, similar to 412, the peg 512 can be movable between a compressed state and an expanded state, and be configured to be biased toward the expanded state. The retaining portion 516 can be configured to retain the peg 512 in the expanded state and the slot 514 can be configured to receive the peg 512 in the compressed state (e.g., the terminal portion 522 can have a diameter that matches the diameter of the peg 512 in the compressed state). Thus, the peg 512 can be press fit into the terminal portion 522 of the slot 514, and an external force (e.g., by rotating the male connection portion 532 relative to the female connection portion 552) can push the peg 512 (in the compressed state) through the slot 514.

According to certain examples, a suture lock (e.g., 300) can be connected to and/or disconnected from an adaptor (e.g., 350) by more than one lock and release mechanisms 410 or 510 having the same or similar peg-slot configurations. Using multiple lock and release mechanisms may increase the strength and alignment of the joint between the suture lock and the adaptor.

As one example, a suture lock and an adaptor can be releasably connected to each other by two that are in diametrically opposite positions when a connecting portion (e.g., 306) of the suture lock is connected to an end portion (e.g., 352) of the adaptor. In other examples, more than two lock and release mechanisms (e.g., 410 or 510) can be used to releasably connect the suture lock to the adaptor. For example, a plurality of lock and release mechanisms can be configured to be equidistant circumferentially when the connecting portion of the suture lock is connected to the end portion of the adaptor.

When multiple lock and release mechanisms 410 or 510 are used to lock/unlock between the suture lock and the adaptor, the pegs and slots (and retaining portions) can be arranged in a variety of ways. In one example, the peg of each lock and release mechanism can be located on the connecting portion (e.g., 306) of the suture lock and the slot of each lock and release mechanism can be located on the end portion (e.g., 352) of the adaptor. In another example, the slot of each lock and release mechanism can be located on the connecting portion of the suture lock and the peg of each lock and release mechanism can be located on the end portion of the adaptor. In yet an alternative example, the pegs of some lock and release mechanisms can be located on the connecting portion of the suture lock and their corresponding slots can be located on the end portion of the adaptor, whereas the pegs of other lock and release mechanisms can be located on the end portion of the adaptor and their corresponding slots can be located on the connecting portion of the suture lock.

### Exemplary Lock and Release Mechanisms Including a Detent

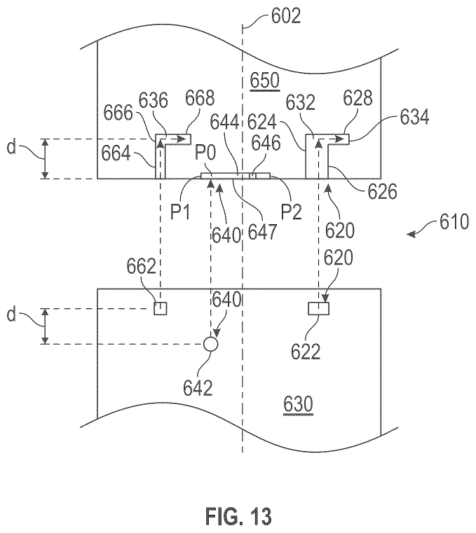

FIG. 13 depicts a lock and release mechanism 610, which can be another example of the lock and release mechanism 310 depicted in FIGS. 10A-10C, through which a suture lock (e.g., 300) can be connected to and/or disconnected from an adaptor (e.g., 350). As described below, the lock and release mechanism 610 does not rely on compression or tension between the suture lock and the adaptor to hold them in place. Instead, the lock and release mechanism 610 uses a detent where a peg or pin can snap fit into to lock/unlock between the suture lock and the adaptor.

FIG. 13 depicts a first component 630 and a second component 650 that can be releasably connected to each other via the lock and release mechanism 610. When connected, at least the connecting portions of the first component 630 and the second component can share a common axial axis 602. In certain examples, the first component 630 can be a suture lock (e.g., 300) and the second component 650 can be an adaptor (e.g., 350). In other examples, the second component 650 can be the suture lock (e.g., 300) and the first component 630 can be the adaptor.

As shown in FIG. 13, the lock and release mechanism 610 includes a first locking mechanism 620 and a second locking mechanism 640. The first locking mechanism 620 can include a first peg 622 and a first slot 624 configured to receive the first peg 622. The second locking mechanism 640 can include a second peg 642 and a second slot 644 configured to receive the second peg 642. The first peg 622 and the second peg 642 can have the same or different shape and/or size.

In the depicted example, both the first peg 622 and the second peg 642 are located on the first component 630, and both the first slot 624 and the second slot 644 are located on the second component 650. In another example, both the first peg 622 and the second peg 642 can be located on the second component 650, and both the first slot 624 and the second slot 644 are located on the first component 630. In yet an alternative example, one of the pegs (e.g., 622 or 642) can be located on the first component 630 and the other peg can be located on the second component 650. Correspondingly, one of the slots (e.g., 624 or 644) can be located on the second component 650 and the other slot can be located on the first component 630.

For example, the first component 630 can have a male portion configured to be inserted into an inner bore of the second component 650. In one example, both pegs 622, 642 can extend radially outwardly from the male portion of the first component 630 and both slots 624, 644 can extend along an inner surface defining the inner bore of the second component 650 (similar to the arrangement of the peg 412 and slot 414 depicted in FIG. 11A). In another example, both pegs 622, 642 can extend radially inwardly from the inner surface of the second component 650 and both slots can extend along an outer surface of the male portion of the first component 630 (similar to the peg 512 and slot 514 depicted in FIG. 12A). In yet another example, one of the pegs (e.g., 622, 642) can extend radially outwardly from the male portion of the first component 630 and the other peg can extend radially inwardly from the inner surface of the second component 650. Correspondingly, one of the slots (e.g., 624, 644) can extend along the inner surface of the second component 650 and the other slot can extend along the outer surface of the first component 630.

Alternatively, the first component 630 can have an inner bore configured to receive a male portion of the second component 650. Similarly, one or both pegs 622, 642 can extend radially outwardly from the male portion of the second component 650 or extend radially inwardly from an inner surface defining the inner bore of the first component 630. Correspondingly, one or both slots 624, 644 can extend along the inner surface of the first component 630 or an outer surface of the male portion of the second component 650.

As depicted in FIG. 13, the first slot 624 can include an axial portion 626 extending parallel to the axial axis 602 and a circumferential portion 628 that is connected to the axial portion 626 at a corner 632 and extends circumferentially about the axial axis 602. Thus, when the first peg 622 is received within the circumferential portion 628 of the first slot 624 (and positioned away from the corner 632), the first component 630 can be rotatable but not axially movable relative to the second component 650 (i.e., axially locked). On the other hand, when the first peg 622 is received within the axial portion 626 of the first slot 624, the first component 630 can be axially movable relative to the second component 650 (i.e., axially unlocked). In other words, by moving the position of the first peg 622 within the first slot 624, the first locking mechanism 620 can lock/unlock axial movement between the first component 630 and the second component 650.

As shown, the second slot 644 can extend circumferentially about the axial axis 602 and have a first end P1 and a second end P2. The second locking mechanism 640 can include a radially recessed detent 646 connected to the second slot 644 and configured to retain the second peg 642. In certain examples, the detent 646 can be formed as a part of the second slot 644. As described below, when the first peg 622 is received within the circumferential portion 628 of the first slot 624, rotation of the first component 630 relative to the second component 650 can move the second peg 642 through the second slot 644 until the second peg 642 snap fits into the detent 646 such that the second slot 644 can inhibit the second peg 642 from moving out of the detent 646.

In certain examples, the second slot 644 is configured to be radially offset from the circumferential portion 628 of the first slot 624 by a distance (d) that is about the same as a length of the axial portion 626 of the first slot 624 such that when the first peg 622 is received in the first slot 624 at the corner 632, the second peg 642 can be received in the second slot 644, e.g., at an initial position PO that is circumferentially spaced away from the detent 646.

Additionally, the circumferential path 647 connecting the detent 646 and the initial position PO extends from the initial position PO in the same angular direction (e.g., clockwise or counterclockwise) as the circumferential portion 628 extends from the corner 632. Thus, when the first peg 622 is received in the first slot 624 at the corner 632, rotation of the first component 630 relative to the second component 650 can simultaneously cause the first peg 622 to move into the circumferential portion 628 (thus axially locked) and cause the second peg 642 to move along the circumferential path 647 toward the detent 646.

In certain examples, the initial position PO can be located at the first end Pl of the second slot 644. In other examples, the initial position PO can be located in a middle portion of the second slot 644. In certain examples, the circumferential length between the initial position PO and the detent 646 is about the same as the circumferential length of the circumferential portion 628. Thus, when the first peg 622 moves to a terminal end 634 of the circumferential portion 628, the second peg 642 can move into the detent 646. In other examples, the circumferential length between the initial position PO and the detent 646 can be smaller than the circumferential length of the circumferential portion 628.

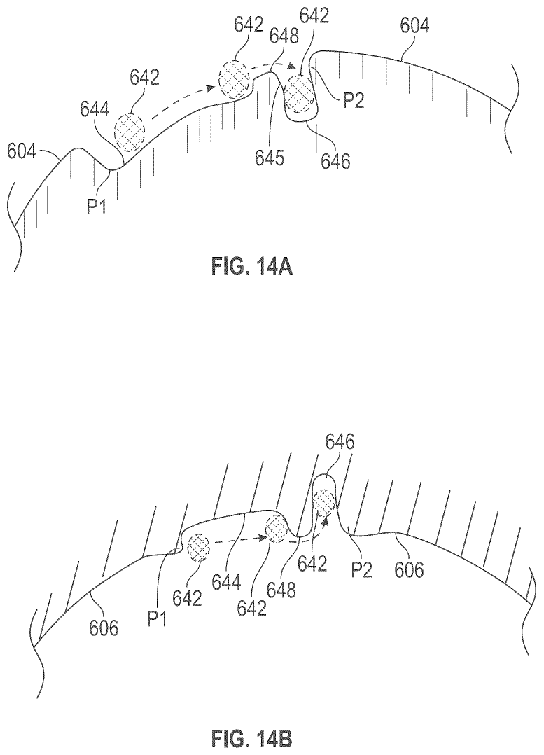

FIG. 14A illustrates an example where the second slot 644 extends along an outer surface 604 of a component, which can be the first component 630 or the second component 650. In this example, the second slot 644 is recessed radially inwardly from the outer surface 604, and the detent 646 is further recessed radially inwardly from the second slot 644 and the outer surface 604. In other words, the detent 646 forms a deeper recess or pit extending radially inwardly from the outer surface 604 than the second slot 644. In the depicted example, the detent 646 is located at the second end P2 of the second slot 644 (i.e., the detent 646 is located between the second slot 644 and non-recessed outer surface 604). In other examples, the detent 646 can be located in a middle portion of the second slot 644 (i.e., the detent 646 is spaced away from the non-recessed outer surface 604).

As illustrated in FIG. 14A, the second peg 642 can be configured to move across the second slot 644 and then drop into and snap fit within the detent 646, e.g., by rotating the first component 630 relative to the second component 650 in a first direction 612. In certain examples, the second peg 642 can be movable between a radially biased position and a radially unbiased position. Specifically, the second peg 642 received in the second slot 644 can be in the radially biased position and the second peg 642 received in the detent 646 can be in the radially unbiased position. Thus, when moving from the second slot 644 to the detent 646, the second peg 642 can return from the radially biased position to the radially unbiased position.

Once retained in the detent 646, the second peg 642 can be inhibited from moving out of the detent 646 (due to the resistance exerted by the wall 645 around the detent 646), thereby resisting rotational movement of the first component 630 relative to the second component 650 (i.e., rotationally locked). On the other hand, the first component 630 can be rotated relative to the second component 650 in a second direction 614 opposite to the first direction 612 with a sufficiently large external force to overcome the resistance of the wall 645 and urge the second peg 642 out of the detent 646 and into the second slot 644, thereby enabling rotational movement of the first component 630 relative to the second component 650 (i.e., rotationally unlocked). In other words, by moving the position of the second peg 642 within the second slot 644 and/or the detent 646, the second locking mechanism 640 can lock/unlock rotational movement between the first component 630 and the second component 650.

As shown in FIG. 14A, in certain examples, the second slot 644 can have a radial protrusion 648 adjacent to the detent 646. The radial protrusion 648 can extend radially outwardly from the remaining part of the second slot 644. In one particular example, the radial protrusion 648 can be configured to have the same radius as the outer surface 604. In other examples, the radial protrusion 648 can have smaller or larger radius than the outer surface 604. Having the radial protrusion 648 adjacent to the detent 646 can increase the radial depth of the detent 646 (i.e., increase the height of the wall 645 separating the detent 646 from the second slot 644), thus an even larger external force is needed to overcome the resistance of the wall 645 to unlock the rotational movement between the first component 630 and the second component 650.

In certain examples, the radial protrusion 648 can be configured to be radially depressible such that the radial protrusion 648 can be radially compressed by the second peg 642 and allow the second peg 642 to move across the radial protrusion 648. For example, the radial protrusion can comprise a deformable material (e.g., rubber, copper, aluminum, nickel titanium alloy, magnesium alloy, etc.). Additionally, or alternatively, the second locking mechanism 640 can include one or more relief channels located underneath the radial protrusion 648 and configured to allow the radial protrusion 648 to be radially compressed when the second peg 642 moves across the radial protrusion 648.

In certain examples, the radial protrusion 648 can be configured to be non-depressible. Instead, the second peg 642 can be configured to be radially depressible or circumferentially deflectable. For example, the second peg 642 can comprise a deformable or deflectable material (e.g., rubber, copper, aluminum, nickel titanium alloy, magnesium alloy, etc.), and/or having a coil or spring structure that allow it to be radially deformed and/or circumferentially deflected by the radial protrusion 648 when moving across the radial protrusion 648.

FIG. 14B illustrates another example where the second slot 644 extends along an inner surface 606 of a component, which can be the first component 630 or the second component 650. In this example, the second slot 644 is recessed radially outwardly from the inner surface 606, and the detent 646 is further recessed radially outwardly from the second slot 644 and the inner surface 606. In other words, the detent 646 forms a deeper recess or pit extending radially outwardly from the inner surface 606 than the second slot 644. In the depicted example, the detent 646 is located at the second end P2 of the second slot 644 (i.e., the detent 646 is located between the second slot 644 and the non-recessed inner surface 606). In other examples, the detent 646 can be located in a middle portion of the second slot 644 (i.e., the detent 646 is spaced away from the non-recessed inner surface 606).

Similar to the example depicted in FIG. 14A, the second peg 642 can be configured to move across the second slot 644 and then snap fit within the detent 646 by rotating the first component 630 relative to the second component 650 in a first direction 616, thus rotationally locking the first component 630 to the second component 650. Likewise, by rotating the first component 630 relative to the second component 650 in a second direction 618 opposite to the first direction 616 and with a sufficiently large external force, the second peg 642 can be moved out of the detent 646 and into the second slot 644, thereby rotationally unlocking the first component 630 from the second component 650.

Similarly, as depicted in FIG. 14B, the second slot 644 can have a radial protrusion 648 adjacent to the detent 646. The radial protrusion 648 can extend radially inwardly from the remaining part of the second slot 644 so as to create a deeper wall around the detent 646. In one particular example, the radial protrusion 648 can be configured to have the same radius as the inner surface 606. In other examples, the radial protrusion 648 can have smaller or larger radius than the inner surface 606. Similarly, the radial protrusion 648 can be configured to be radially depressible such that the radial protrusion 648 can be radially compressed by the second peg 642 when moving the second peg 642 across the radial protrusion 648. Additionally, or alternatively, the second peg 642 can be configured to be radially depressible or circumferentially deflectable so that the second peg 642 can be radially deformed and/or circumferentially deflected by the radial protrusion 648 when moving across the radial protrusion 648.

In certain examples, as depicted in FIG. 13, the first locking mechanism 620 can optionally include a third peg 662 and a third slot 664 configured to receive the third peg 662. In the depicted example, the third peg 662 is located on the first component 630 and the third slot 664 is located on the second component 650. Alternatively, the third peg 662 can be located on the second component 650 and the third slot 664 can be located on the first component 630. Similar to the first and second pegs 622, 642, the third peg 662 can extend radially outwardly from an outer surface or extend radially inwardly from an inner surface, depending on the component on which the third peg is located has a male portion configured to be inserted into an inner bore of the other component or has an inner bore configured to receive a male portion of the other component. Correspondingly, the third slot 664 can extend along an inner surface or an outer surface of the first or second component.

The third slot 664 can have a similar configuration as the first slot 624. For example, the third slot 664 can include an axial portion 666 extending parallel to the axial axis 602 and a circumferential portion 668 that is connected to the axial portion 666 at a corner 636 and extends circumferentially about the axial axis 602. In certain examples, the axial portion 666 of the third slot 664 can have about the same length as the axial portion 626 of the first slot 624 such that the circumferential portion 668 of the third slot 664 is axially aligned with the circumferential portion 628 of the first slot 624. Thus, when the first peg 622 is received in the first slot 624 at the corner 632 (and the second peg 642 is received in the second slot 644), the third peg 662 is also received in the third slot 664 at the corner 636.

As shown, when the first component 630 is connected to the second component 650, the second slot 644 can be configured to be positioned between the first slot 624 and the third slot 664. Similarly, the first peg 622 and the third peg 662 can be positioned at circumferentially opposite sides of the second peg 642. In certain examples, the positions of the first peg 622 and the third slot 662 can be symmetric about the second peg 642. In other examples, the positions of the first peg 622 and the third slot 662 can be asymmetric about the second peg 642.

In certain examples, the first peg 622 and the third peg 662 can have different sizes. For example, as illustrated in FIG. 13, the first peg 622 can have a larger width or diameter (measured perpendicularly to the axial axis 602) than the third peg 662 (or vice versa). The axial portion 666 of the third slot 664 can have about the same width or diameter as the third peg 662, and the axial portion 626 of first slot 624 can have about the same width or diameter as the first peg 622. Accordingly, the axial portion 666 of the third slot 664 can have a smaller width or diameter than the axial portion 626 of first slot 624. Thus, the asymmetric size of the first peg 622 and the third peg 662 (and the asymmetric size of the axial portions 666, 626) can prevent inadvertent insertion of the first peg 622 into the axial portion 666 of the third slot 664 (or vice versa). On the other hand, other dimensions (e.g., height measured along the axial axis 602) of the first peg 622 and the third peg 662 can be the same or different. Accordingly, the width or diameter of the circumferential portion 628 of the first slot 624 and the circumferential portion 668 of the third slot 664 can be the same or different.

In certain examples, the first peg 622 and the third peg 662 can have different shapes. For example, as illustrated in FIG. 13, the first peg 622 can have a rectangular cross-section whereas the third peg 662 can have a square-shaped cross-section. In other examples, the cross-sections of the first peg 622 and the third peg 662 can have other shapes, e.g., circular, oval, triangle, hexagonal, etc.

In other examples, the first peg 622 and the third peg 662 can have about the same shape and size.

In certain examples, the first locking mechanism 620 and the second locking mechanism 640 defines a locking set, and the lock and release mechanism 610 can include a plurality of locking sets. For example, the first component 630 and the second component 650 can be releasably connected to each other by two locking sets in diametrically opposite positions.

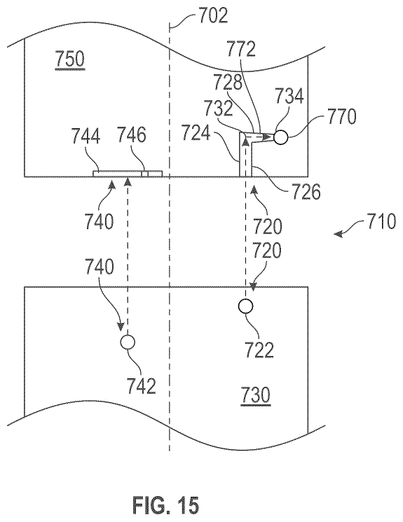

FIG. 15 shows a lock and release mechanism 710, which can be another example of the lock and release mechanism 310 depicted in FIGS. 10A-10C, through which a first component 730 (e.g., a suture lock or an adaptor) and a second component 750 (e.g., an adaptor or a suture lock) can be releasably connected to each other.

Similar to 610, the lock and release mechanism 710 includes a first locking mechanism 720 and a second locking mechanism 740. The second locking mechanism 740 can be configured to be identical to the second locking mechanism 640 described above with reference to FIGS. 13 and 14A-14B. For example, the second locking mechanism 740 can include a second peg 742, a second slot 744 configured to receive the second peg 742, and a radially recessed detent 746 connected to the second slot 744. The second peg 742 can be configured to snap fit within the detent 746. Thus, similar to 640, the second locking mechanism 740 can lock/unlock rotational movement between the first component 730 and the second component 750 about an axial axis 702 of the first component 730 and the second component 750.

Similar to 620, the first locking mechanism 720 can include a first peg 722 and a first slot 724 configured to receive the first peg 722, and the first slot 724 can include an axial portion 726 and a circumferential portion 728 connected to the axial portion 726 at a corner 732. Likewise, by moving the position of the first peg 722 within the first slot 724, the first locking mechanism 720 can lock/unlock axial movement between the first component 730 and the second component 750.

The relative positions and orientation of the first and second pegs 722, 742 and the first and second slots 724, 744 can be configured to be the same as the corresponding parts depicted in FIG. 13. Thus, when the first peg 722 is received in the first slot 724 at the corner 732, the second peg 742 can be received in the second slot 744.

Different from 620, as shown in FIG. 15, the first locking mechanism 720 can further include a retaining portion 770 connected to a terminal end 734 of the circumferential portion 728 and configured to retain the first peg 722. In certain examples, the first peg 722 can be configured to be retained in the retaining portion 770 when the second peg 742 snap fits into the detent 746.

In certain examples, the first peg 722, the first slot 724, and the retaining portion 770 can be configured to be similar to the peg 412 (or 512), the slot 414 (or 514), and the retaining portion 416 (or 516) described above with reference to FIG. 11A (or FIG. 12A). Thus, in additional to axial locking/unlocking, the first locking mechanism 720 can provide additional, thus further enhancing the rotational locking/unlocking provided by the second locking mechanism 740.

For example, the circumferential portion 728 can include a terminal portion 772 adjacent to the retaining portion 770. The retaining portion 770 can have a larger diameter and/or cross-sectional area than that of the terminal end 734 and the terminal portion 772, and the first peg 722 can form an interference fit with the terminal portion 772. In certain examples, the circumferential portion 728 can have a tapered shape such that the diameter of the circumferential portion 728 can progressively decreases from the corner 732 to the terminal end 734 and/or the retaining portion 770.

Similar to 422 and 522 described above, the terminal portion 772 can be deflectable (e.g., by incorporating one or more relief channels around the terminal portion 772 and/or using a deformable material around the terminal portion 772), such that the terminal portion 772 can be dilated when the first peg 722 is pushed through the terminal portion 772 (e.g., by rotating the first component 730 relative to the second component 750).

Alternatively, similar to 412 and 512, the first peg 722 can be movable between a biased compressed state and an unbiased expanded state. For example, the retaining portion 770 can be configured to retain the first peg 722 in the expanded state and the first slot 724 can be configured to receive the first peg 722 in the compressed state. Thus, the first peg 722 can be press fit into the terminal portion 772 of the first slot 724, and an external force (e.g., by rotating the first component 730 relative to the second component 750) can push the first peg 722 (in the compressed state) through the first slot 724.

Although not shown, in certain examples, the first locking mechanism 720 can further include a third peg and a third slot similar to 662 and 664 described above with reference to FIG. 14A. In certain examples, the third slot can be further connected with another retaining portion configured to retain the third peg. For example, the third peg, the third slot, and the retaining portion connected thereof can be configured to be similar to the peg 412 (or 512/722), the slot 414 (or 514/724), and the retaining portion 416 (or 516/770) described above, so as to further enhance the rotational locking/unlocking provided by the second locking mechanism 740.

### ADDITIONAL EXAMPLES OF THE DISCLOSED TECHNOLOGY

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A handle assembly for a delivery apparatus configured to deliver a prosthetic implant, the handle assembly comprising: a first component comprising a male connection portion, wherein the male connection portion comprises a main body with a pin extending radially outwardly from the main body; a second component comprising a female connection portion configured to receive the male connection portion of the first component, wherein the female connection portion comprises an inner surface defining an inner bore, a slot extending along the inner surface, and a retaining portion connected to the slot and configured to receive the pin, wherein the first component and the second component are movable between a locked configuration and a release configuration by inserting the main body into the inner bore and rotating the male connection portion relative to the female connection portion less than one revolution about an axial axis of the main body, wherein in the locked configuration, the pin is positioned within the retaining portion and the slot is configured to resist rotational movement of male connection portion relative to the female connection portion, and wherein in the release configuration, the pin is positioned within the slot.

Example 2. The handle assembly of any example herein, particular example 1, wherein the first component comprises a spool configured to connect to a release suture which extends through the inner bore of the second component and is tied to the prosthetic implant.

Example 3. The handle assembly of any example herein, particular example 1, wherein the second component comprises a spool configured to connect to a release suture which extends through a lumen of the first component and is tied to the prosthetic implant.

Example 4. The handle assembly of any example herein, particularly any one of examples 1-3, wherein the slot comprises an axial portion extending parallel to the axial axis and a circumferential portion connected to the axial portion and extending circumferentially about the axial axis, wherein the retaining portion is connected to a terminal end of the circumferential portion.

Example 5. The handle assembly of any example herein, particular example 4, wherein the circumferential portion comprises a deflectable portion adjacent to the retaining portion such that the deflectable portion can be dilated when the pin is received in the deflectable portion.

Example 6. The handle assembly of any example herein, particular example 5, wherein the female connection portion comprises at least one relief channel extending parallel to the circumferential portion and configured to allow the deflectable portion to axially dilate when the pin moves through the deflectable portion.

Example 7. The handle assembly of any example herein, particularly any one of examples 5-6, wherein the female connection portion comprises a deformable material around the deflectable portion such that when the pin moves through the deflectable portion, the pin can compress the deformable material and axially dilate the deflectable portion.

Example 8. The handle assembly of any example herein, particular example 4, wherein the pin is movable between a compressed state and an expanded state, the pin being biased toward the expanded state, wherein the retaining portion is configured to retain the pin in the expanded state and the slot is configured to receive the pin in the compressed state.

Example 9. The handle assembly of any example herein, particular example 8, wherein the slot has a substantially constant diameter that matches a diameter of the pin in the compressed state.

Example 10. The handle assembly of any example herein, particularly any one of examples 1-3, wherein the pin is a second pin and the slot is a second slot, wherein the male connection portion comprises a first pin extending radially outwardly from the main body, wherein the first pin is axially offset from the second pin by a predefined distance, wherein the female connection portion comprises a first slot configured to receive the first pin.

Example 11. The handle assembly of any example herein, particular example 10, wherein the first slot comprises an axial portion extending parallel to the axial axis and a circumferential portion extending circumferentially about the axial axis, wherein the axial portion has an axial length equal to the predefined distance.

Example 12. The handle assembly of any example herein, particular example 11, wherein the male connection portion comprises a third pin extending radially outwardly from the main body, wherein the female connection portion comprises a third slot configured to receive the third pin.

Example 13. The handle assembly of any example herein, particular example 12, wherein the first pin and the third pin are positioned at circumferentially opposite sides of the second pin.

Example 14. The handle assembly of any example herein, particularly any one of examples 12-13, wherein the third slot comprises an axial portion extending parallel to the axial axis and a circumferential portion extending circumferentially about the axial axis, wherein the circumferential portion of the third slot is axially aligned with the circumferential portion of the first slot.

Example 15. The handle assembly of any example herein, particularly any one of examples 12-14, wherein the first pin and the third pin have different sizes.

Example 16. The handle assembly of any example herein, particularly any one of examples 12-15, wherein the first pin and the third pin have different shapes.

Example 17. The handle assembly of any example herein, particularly any one of examples 1-16, wherein the retaining portion comprises a radially recessed detent relative to the slot and the inner surface the female connection portion.

Example 18. The handle assembly of any example herein, particular example 17, wherein the slot comprises a radial protrusion adjacent to the radially recessed detent.

Example 19. The handle assembly of any example herein, particular example 18, wherein the radial protrusion is configured to be radially depressible such that the radial protrusion can be radially compressed to allow the pin to move across the radial protrusion.

Example 20. The handle assembly of any example herein, particularly any one of examples 18-19, wherein the pin is configured to be deformable such that the pin can be radially compressed when moving across the radial protrusion.

Example 21. A handle assembly for a delivery apparatus configured to deliver a prosthetic implant, the handle assembly comprising: a first component comprising a female connection portion, wherein the female connection portion comprises an inner surface defining an inner bore and a pin extending radially inwardly from the inner surface; a second component comprising a male connection portion configured to be inserted into the inner bore of the female connection portion, wherein the male connection portion comprises an outer surface, a slot extending along the outer surface, and a retaining portion connected to the slot and configured to receive the pin, wherein the first component and the second component are movable between a locked configuration and a release configuration by inserting the male connection portion into the inner bore and rotating the male connection portion relative to the female connection portion less than one revolution about an axial axis of the inner bore, wherein in the locked configuration, the pin is positioned within the retaining portion and the slot is configured to resist rotational movement of male connection portion relative to the female connection portion, and wherein in the release configuration, the pin is positioned within the slot.

Example 22. The handle assembly of any example herein, particular example 21, wherein the second component comprises a spool configured to connect to a release suture which extends through the inner bore of the first component and is tied to the prosthetic implant.

Example 23. The handle assembly of any example herein, particular example 21, wherein the first component comprises a spool configured to connect to a release suture which extends through a lumen of the second component and is tied to the prosthetic implant.

Example 24. The handle assembly of any example herein, particularly any one of examples 21-23, wherein the slot comprises an axial portion extending parallel to the axial axis and a circumferential portion connected to the axial portion and extending circumferentially about the axial axis, wherein the retaining portion is connected to a terminal end of the circumferential portion.

Example 25. The handle assembly of any example herein, particular example 24, wherein the circumferential portion comprises a deflectable portion adjacent to the retaining portion such that the deflectable portion can be dilated when the pin is received in the deflectable portion.

Example 26. The handle assembly of any example herein, particular example 25, wherein the male connection portion comprises at least one relief channel extending parallel to the circumferential portion and configured to allow the deflectable portion to axially dilate when the pin moves through the deflectable portion.

Example 27. The handle assembly of any example herein, particularly any one of examples 25-26, wherein the male connection portion comprises a deformable material around the deflectable portion such that when the pin moves through the deflectable portion, the pin can compress the deformable material and axially dilate the deflectable portion.

Example 28. The handle assembly of any example herein, particular example 24, wherein the pin is movable between a compressed state and an expanded state, the pin being biased toward the expanded state, wherein the retaining portion is configured to retain the pin in the expanded state and the slot is configured to receive the pin in the compressed state.

Example 29. The handle assembly of any example herein, particular example 28, wherein the slot has a substantially constant diameter that matches a diameter of the pin in the compressed state.

Example 30. The handle assembly of any example herein, particularly any one of examples 21-23, wherein the pin is a second pin and the slot is a second slot, wherein the female connection portion comprises a first pin extending radially inwardly from the inner surface, wherein the first pin is axially offset from the second pin by a predefined distance, wherein the male connection portion comprises a first slot configured to receive the first pin.

Example 31. The handle assembly of any example herein, particular example 30, wherein the first slot comprises an axial portion extending parallel to the axial axis and a circumferential portion extending circumferentially about the axial axis, wherein the axial portion has an axial length equal to the predefined distance.

Example 32. The handle assembly of any example herein, particular example 31, wherein the female connection portion comprises a third pin extending radially inwardly from the inner surface, wherein the male connection portion comprises a third slot configured to receive the third pin.

Example 33. The handle assembly of any example herein, particular example 32, wherein the first pin and the third pin are positioned at circumferentially opposite sides of the second pin.

Example 34. The handle assembly of any example herein, particularly any one of examples 32-33, wherein the third slot comprises an axial portion extending parallel to the axial axis and a circumferential portion extending circumferentially about the axial axis, wherein the circumferential portion of the third slot is axially aligned with the circumferential portion of the first slot.

Example 35. The handle assembly of any example herein, particularly any one of examples 32-34, wherein the first pin and the third pin have different sizes.

Example 36. The handle assembly of any example herein, particularly any one of examples 32-35, wherein the first pin and the third pin have different shapes.

Example 37. The handle assembly of any example herein, particularly any one of examples 21-36, wherein the retaining portion comprises a radially recessed detent relative to the slot and the outer surface the male connection portion.

Example 38. The handle assembly of any example herein, particular example 37, wherein the slot comprises a radial protrusion adjacent to the radially recessed detent.

Example 39. The handle assembly of any example herein, particular example 38, wherein the radial protrusion is configured to be radially depressible such that the radial protrusion can be radially compressed to allow the pin to move across the radial protrusion.

Example 40. The handle assembly of any example herein, particularly any one of examples 38-39, wherein the pin is configured to be deformable such that the pin can be radially compressed when moving across the radial protrusion.

Example 41. A handle assembly for a delivery apparatus configured to deliver a prosthetic implant, the handle assembly comprising: a suture lock configured to connect to a release suture which is tied to the prosthetic implant; and an adaptor comprising a lumen configured for the release suture to extend through and an end portion configured to be releasably connected to a connecting portion of the suture lock via a lock and release mechanism, wherein the lock and release mechanism comprises a peg and a slot configured to receive the peg, wherein the slot comprises a deflectable portion and a retaining portion connected to the deflectable portion, wherein the retaining portion is configured to retain the peg, wherein the deflectable portion has a smaller diameter than the peg, wherein rotation of the suture lock relative to the adaptor in a first direction is configured to move the peg through the deflectable portion and lock into the retaining portion such that the deflectable portion inhibits the peg from moving out of the retaining portion, wherein rotation of the suture lock relative to the adaptor in a second direction opposite to the first direction is configured to move the peg out of the retaining portion through the deflectable portion.

Example 42. The handle assembly of any example herein, particular example 41, wherein when connected, the connecting portion of the suture lock and the end portion of the adaptor are coaxial about an axial axis.

Example 43. The handle assembly of any example herein, particular example 42, wherein the slot comprises a first slot portion extending parallel to the axial axis and a second slot portion connecting to the first slot portion at a corner and extending circumferentially about the axial axis, wherein the second slot portion comprises the deflectable portion and the retaining portion is located at a terminal end of the second slot portion.

Example 44. The handle assembly of any example herein, particular example 43,

wherein the second slot portion comprises a cutout window extending radially through a wall of the end portion of the adaptor or a wall of the connection portion of the suture lock so as to expose the peg when the peg is moved to the cutout window.

Example 45. The handle assembly of any example herein, particular example 44, wherein the cutout window extends from the retaining portion to a cutout edge that is spaced apart from the corner.

Example 46. The handle assembly of any example herein, particularly any one of examples 44-45, wherein the lock and release mechanism further comprises at least one relief channel extending parallel to the second slot portion, wherein the relief channel comprises an opening extending radially through the wall of the end portion of the adaptor or the wall of the connection portion of the suture lock such that when the peg moves through the deflectable portion, the deflectable portion can axially dilate toward the relief channel.

Example 47. The handle assembly of any example herein, particular example 46, wherein a circumferential length of the relief channel is the same as or larger than a circumferential length of the cutout window.

Example 48. The handle assembly of any example herein, particularly any one of examples 46-47, wherein the lock and release mechanism comprises two relief channels extending parallel to and located on both sides of the second slot portion.

Example 49. The handle assembly of any example herein, particularly any one of examples 43-48, wherein a diameter of the second slot portion progressively decreases from the corner to the deflectable portion.

Example 50. The handle assembly of any example herein, particularly any one of examples 43-49, wherein a wall through which the slot is formed comprises a deformable material around the second slot portion such that when the peg moves through the deflectable portion, the peg can compress the deformable material and axially dilate the deflectable portion.

Example 51. The handle assembly of any example herein, particularly any one of examples 41-50, wherein the peg is located on the connecting portion of the suture lock and the slot is located on the end portion of the adaptor.

Example 52. The handle assembly of any example herein, particular example 51, wherein the peg extends radially outwardly from the connecting portion of the suture lock, wherein the connecting portion of the suture lock is configured to be inserted into an inner bore of the end portion of the adaptor.

Example 53. The handle assembly of any example herein, particular example 51, wherein the peg extends radially inwardly from the connecting portion of the suture lock, wherein the end portion of the adaptor is configured to be inserted into an inner bore of the connecting portion of the suture lock.

Example 54. The handle assembly of any example herein, particularly any one of examples 41-50, wherein the slot is located on the connecting portion of the suture lock and the peg is located on the end portion of the adaptor.

Example 55. The handle assembly of any example herein, particular example 54, wherein the peg extends radially outwardly from the end portion of the adaptor, wherein the end portion of the adaptor is configured to be inserted into an inner bore of the connecting portion of the suture lock.

Example 56. The handle assembly of any example herein, particular example 54, wherein the peg extends radially inwardly from the end portion of the adaptor, wherein the connecting portion of the suture lock is configured to be inserted into an inner bore of the end portion of the adaptor.

Example 57. The handle assembly of any example herein, particularly any one of examples 41-56, wherein the lock and release mechanism is one of a plurality of lock and release mechanisms of the handle assembly.

Example 58. The handle assembly of any example herein, particular example 57 comprises two lock and release mechanisms that are in diametrically opposite positions when the connecting portion of the suture lock is connected to the end portion of the adaptor.

Example 59. The handle assembly of any example herein, particularly any one of examples 57-58, wherein the peg of each lock and release mechanism is located on the connecting portion of the suture lock and the slot of each lock and release mechanism is located on the end portion of the adaptor.

Example 60. The handle assembly of any example herein, particularly any one of examples 57-58, wherein the slot of each lock and release mechanism is located on the connecting portion of the suture lock and the peg of each lock and release mechanism is located on the end portion of the adaptor.

Example 61. A handle assembly for a delivery apparatus configured to deliver a prosthetic implant, the handle assembly comprising: a suture lock configured to connect to a release suture which is tied to the prosthetic implant; and an adaptor comprising a lumen configured for the release suture to extend through and an end portion configured to be releasably connected to a connecting portion of the suture lock via a lock and release mechanism, wherein when connected, the connecting portion of the suture lock and the end portion of the adaptor are coaxial about an axial axis, wherein the lock and release mechanism comprises a peg and a slot configured to receive the peg, wherein the peg is movable between a compressed state and an expanded state, the peg being biased toward the expanded state, wherein the slot comprises a track portion and a retaining portion connected to the track portion, wherein the retaining portion is configured to retain the peg in the expanded state, wherein the track portion is configured to receive the peg in the compressed state and has a diameter smaller than the peg in the expanded state, wherein rotation of the suture lock relative to the adaptor in a first direction is configured to move the peg in the compressed state through the track portion and into the retaining portion such that the peg is locked therein in the expanded state and the track portion inhibits the peg from moving out of the retaining portion, wherein rotation of the suture lock relative to the adaptor in a second direction opposite to the first direction is configured to move the peg from the expanded state to the compressed state and move the peg out of the retaining portion through the track portion.

Example 62. The handle assembly of any example herein, particular example 61, wherein the track portion comprises a first track portion extending parallel to the axial axis and a second track portion connecting to the first track portion at a corner and extending circumferentially about the axial axis, wherein the retaining portion is located at a terminal end of the second track portion.

Example 63. The handle assembly of any example herein, particularly any one of examples 61-62, wherein the track portion has a substantially constant diameter that matches a diameter of the peg in the compressed state.

Example 64. The handle assembly of any example herein, particularly any one of examples 61-63, wherein the peg is located on the connecting portion of the suture lock and the slot is located on the end portion of the adaptor.

Example 65. The handle assembly of any example herein, particular example 64, wherein the peg extends radially outwardly from the connecting portion of the suture lock, wherein the connecting portion of the suture lock is configured to be inserted into an inner bore of the end portion of the adaptor.

Example 66. The handle assembly of any example herein, particular example 64, wherein the peg extends radially inwardly from the connecting portion of the suture lock, wherein the end portion of the adaptor is configured to be inserted into an inner bore of the connecting portion of the suture lock.

Example 67. The handle assembly of any example herein, particularly any one of examples 61-63, wherein the slot is located on the connecting portion of the suture lock and the peg is located on the end portion of the adaptor.

Example 68. The handle assembly of any example herein, particular example 67, wherein the peg extends radially outwardly from the end portion of the adaptor, wherein and the end portion of the adaptor is configured to be inserted into an inner bore of the connecting portion of the suture lock.

Example 69. The handle assembly of any example herein, particular example 67, wherein the peg extends radially inwardly from the end portion of the adaptor, wherein the connecting portion of the suture lock is configured to be inserted into an inner bore of the end portion of the adaptor.

Example 70. The handle assembly of any example herein, particularly any one of examples 61-69, wherein the lock and release mechanism is one of a plurality of lock and release mechanisms of the handle assembly that are equidistant circumferentially when the connecting portion of the suture lock is connected to the end portion of the adaptor.

Example 71. A handle assembly for a delivery apparatus configured to deliver a prosthetic implant, the handle assembly comprising: a suture lock configured to connect to a release suture which is tied to the prosthetic implant; and an adaptor comprising a lumen configured for the release suture to extend through and an end portion configured to be releasably connected to a connecting portion of the suture lock via a lock and release mechanism, wherein when connected, the connecting portion of the suture lock and the end portion of the adaptor are coaxial about an axial axis, wherein the lock and release mechanism comprises a peg and a slot configured to receive the peg, wherein the slot comprises an axial portion extending parallel to the axial axis and a circumferential portion connected to the axial portion and extending circumferentially about the axial axis, wherein the lock and release mechanism further comprises a retaining portion connected to a terminal end of the circumferential portion and configured to retain the peg, wherein the retaining portion has a larger diameter than the circumferential portion, wherein rotation of the suture lock relative to the adaptor in a first direction is configured to move the peg through the circumferential portion and locked into the retaining portion such that the circumferential portion inhibits the peg from moving out of the retaining portion, wherein rotation of the suture lock relative to the adaptor in a second direction opposite to the first direction is configured to move the peg out of the retaining portion through the circumferential portion.

Example 72. The handle assembly of any example herein, particular example 71, wherein the circumferential portion comprises a deflectable portion adjacent to the retaining portion such that the deflectable portion can be dilated when the peg is received in the deflectable portion.

Example 73. The handle assembly of any example herein, particular example 72, wherein the lock and release mechanism further comprises one or more relief channels extending parallel to the circumferential portion and configured to allow the deflectable portion to axially dilate when the peg moves through the deflectable portion.

Example 74. The handle assembly of any example herein, particularly any one of examples 72-73, wherein a wall through which the slot is formed comprises a deformable material around the deflectable portion such that when the peg moves through the deflectable portion, the peg can compress the deformable material and axially dilate the deflectable portion.

Example 75. The handle assembly of any example herein, particular example 71, wherein the peg is movable between a compressed state and an expanded state, the peg being biased toward the expanded state, wherein the retaining portion is configured to retain the peg in the expanded state and the slot is configured to receive the peg in the compressed state.

Example 76. The handle assembly of any example herein, particular example 75, wherein the slot has a substantially constant diameter that matches a diameter of the peg in the compressed state.

Example 77. The handle assembly of any example herein, particularly any one of examples 71-76, wherein the peg is located on the connecting portion of the suture lock and the slot is located on the end portion of the adaptor.

Example 78. The handle assembly of any example herein, particularly any one of examples 71-76, wherein the slot is located on the connecting portion of the suture lock and the peg is located on the end portion of the adaptor.

Example 79. The handle assembly of any example herein, particularly any one of examples 71-78, wherein the connecting portion of the suture lock is configured to be inserted into an inner bore of the end portion of the adaptor.

Example 80. The handle assembly of any example herein, particularly any one of examples 71-78, wherein the end portion of the adaptor is configured to be inserted into an inner bore of the connecting portion of the suture lock.

Example 81. A handle assembly for a delivery apparatus configured to deliver a prosthetic implant, the handle assembly comprising: a suture lock configured to connect to a release suture which is tied to the prosthetic implant; and an adaptor comprising a lumen configured for the release suture to extend through and an end portion configured to be releasably connected to a connecting portion of the suture lock via a lock and release mechanism, wherein when connected, the connecting portion of the suture lock and the end portion of the adaptor are coaxial about an axial axis, wherein the lock and release mechanism comprises a first locking mechanism and a second locking mechanism, wherein the first locking mechanism comprises a first peg and a first slot configured to receive the first peg, wherein when the first peg is received within a circumferential portion of the first slot, the suture lock is configured to be rotatable but not axially movable relative to the adaptor, wherein the second locking mechanism comprises a second peg and a second slot configured to receive the second peg, the second slot extending circumferentially about the axial axis, wherein a radially recessed detent is connected to the second slot and configured to retain the second peg, wherein when the first peg is received within the circumferential portion of the first slot, rotation of the suture lock relative to the adaptor in a first direction is configured to move the second peg through the second slot until the second peg snap fits into the radially recessed detent such that the second slot inhibits the second peg from moving out of the radially recessed detent, wherein rotation of the suture lock relative to the adaptor in a second direction opposite to the first direction is configured to move the second peg out of the radially recessed detent through the second slot.

Example 82. The handle assembly of any example herein, particular example 81, wherein the first slot comprises an axial portion extending parallel to the axial axis, wherein the circumferential portion is connected to the axial portion at a corner and extends circumferentially about the axial axis.

Example 83. The handle assembly of any example herein, particular example 82, wherein the second slot is radially offset from the circumferential portion of the first slot by a distance that is about the same as a length of the axial portion of the first slot such that the second peg is received in the second slot when the first peg is received in the first slot at the corner.

Example 84. The handle assembly of any example herein, particularly any one of examples 81-83, wherein the first locking mechanism further comprises a retaining portion connected to a terminal end of the circumferential portion and configured to retain the first peg, wherein the terminal end of the circumferential portion has a first cross-sectional area, and the retaining portion has a second cross-sectional area that is larger than the first cross-sectional area.

Example 85. The handle assembly of any example herein, particular example 84, wherein the first peg is configured to be retained in the retaining portion when the second peg snap fits into the radially recessed detent.

Example 86. The handle assembly of any example herein, particularly any one of examples 84-85, wherein the circumferential portion comprises a deflectable portion adjacent to the retaining portion such that the deflectable portion can be dilated when the first peg is received in the deflectable portion.

Example 87. The handle assembly of any example herein, particular example 86, wherein the first locking mechanism further comprises one or more relief channels extending parallel to the circumferential portion and configured to allow the deflectable portion to axially dilate when the first peg moves through the deflectable portion.

Example 88. The handle assembly of any example herein, particularly any one of examples 86-87, wherein a wall through which the first slot is formed comprises a deformable material around the deflectable portion such that when the first peg moves through the deflectable portion, the first peg can compress the deformable material and axially dilate the deflectable portion.

Example 89. The handle assembly of any example herein, particularly any one of examples 84-85, wherein the first peg is movable between a compressed state and an expanded state, the first peg being biased toward the expanded state, wherein the retaining portion is configured to retain the first peg in the expanded state and the first slot is configured to receive the first peg in the compressed state.

Example 90. The handle assembly of any example herein, particularly any one of examples 81-89, wherein first locking mechanism further comprises a third peg and a third slot configured to receive the third peg, wherein the third slot comprises a circumferential portion that is axially aligned with the circumferential portion of the first slot.

Example 91. The handle assembly of any example herein, particular example 90, wherein the second slot is positioned between the first slot and the third slot when the connecting portion of the suture lock is connected to the end portion of the adaptor.

Example 92. The handle assembly of any example herein, particularly any one of examples 90-91, wherein the third slot comprises an axial portion extending parallel to the axial axis and connected to the circumferential portion of the third slot.

Example 93. The handle assembly of any example herein, particularly any one of examples 90-92, wherein the first peg and the third peg have different sizes.

Example 94. The handle assembly of any example herein, particularly any one of examples 90-93, wherein the first peg and the third peg have different shapes.

Example 95. The handle assembly of any example herein, particularly any one of examples 90-92, wherein the first peg and the third peg have about the same shape and size.

Example 96. The handle assembly of any example herein, particularly any one of examples 81-95, wherein the second slot comprises a radial protrusion adjacent to the radially recessed detent.

Example 97. The handle assembly of any example herein, particular example 96, wherein the radial protrusion is configured to be radially depressible such that the radial protrusion can be radially compressed to allow the second peg to move across the radial protrusion.

Example 98. The handle assembly of any example herein, particular example 97, wherein the radial protrusion comprises a deformable material.

Example 99. The handle assembly of any example herein, particularly any one of examples 97-98, where the second locking mechanism comprises one or more relief channels located underneath the radial protrusion and configured to allow the radial protrusion to be radially compressed when the second peg moves across the radial protrusion.

Example 100. The handle assembly of any example herein, particularly any one of examples 96-99, wherein the second peg is configured to be deformable such that the second peg can be radially compressed when moving across the radial protrusion.

Example 101. The handle assembly of any example herein, particularly any one of examples 81-100, wherein the first peg and the second peg are located on the connecting portion of the suture lock, and the first slot and the second slot are located on the end portion of the adaptor.

Example 102. The handle assembly of any example herein, particularly any one of examples 81-100, wherein the first slot and the second slot are located on the connecting portion of the suture lock, and the first peg and the second peg are located on the end portion of the adaptor.

Example 103. The handle assembly of any example herein, particularly any one of examples 81-100, wherein the first peg and the second slot are located on the connecting portion of the suture lock, and the second peg and the first slot are located on the end portion of the adaptor.

Example 104. The handle assembly of any example herein, particularly any one of examples 81-100, wherein the first slot and the second peg are located on the connecting portion of the suture lock, and the first peg and the second slot are located on the end portion of the adaptor.

Example 105. The handle assembly of any example herein, particularly any one of examples 81-104, wherein the connecting portion of the suture lock is configured to be inserted into an inner bore of the end portion of the adaptor.

Example 106. The handle assembly of any example herein, particularly any one of examples 81-104, wherein the end portion of the adaptor is configured to be inserted into an inner bore of the connecting portion of the suture lock.

Example 107. The handle assembly of any example herein, particularly any one of examples 81-106, wherein the first locking mechanism and the second locking mechanism defines a locking set, wherein the handle assembly comprises a plurality of locking sets.

Example 108. The handle assembly of any example herein, particular example 107, wherein the suture lock comprises two locking sets that are in diametrically opposite positions when the connecting portion of the suture lock is connected to the end portion of the adaptor.

Example 109. A delivery apparatus configured to deliver a prosthetic implant, the delivery apparatus comprising: a handle assembly according to any example herein, particularly any one of examples 41-108; and a delivery sheath extending distally from the handle assembly.

Example 110. The delivery apparatus of any example herein, particular example 109, wherein the adaptor comprises a straight section and a branch extending sideway from the straight section, wherein the end portion of the adaptor that is releasably connected to the connecting portion of the suture lock is located on the branch.

Example 111. A method of implanting a prosthetic implant, the method comprising: assembling a handle assembly according to any example herein, particularly any one of examples 41-108, wherein the assembling comprises rotating the suture lock relative to the adaptor in the first direction so as to connect the suture lock to the adaptor.

Example 112. The method of any example herein, particular example 111, further comprising rotation of the suture lock relative to the adaptor in the second direction so as to disconnect the suture lock from the adaptor.

Example 113. The method of any example herein, particularly any one of examples 111-112, further comprising cutting the release suture.

In view of the many possible examples to which the principles of the disclosed technology may be applied, it should be recognized that the illustrated examples are only preferred examples of the technology and should not be taken as limiting the scope of the disclosure. Rather, the scope of the claimed subject matter is defined by the following claims and their equivalents.

1. A handle assembly for a delivery apparatus configured to deliver a prosthetic implant, the handle assembly comprising:
a first component comprising a male connection portion, wherein the male connection portion comprises a main body with a pin extending radially outwardly from the main body;
a second component comprising a female connection portion configured to receive the male connection portion of the first component, wherein the female connection portion comprises an inner surface defining an inner bore, a slot extending along the inner surface, and a retaining portion connected to the slot and configured to receive the pin,
wherein the first component and the second component are movable between a locked configuration and a release configuration by inserting the main body into the inner bore and rotating the male connection portion relative to the female connection portion less than one revolution about an axial axis of the main body,
wherein in the locked configuration, the pin is positioned within the retaining portion and the slot is configured to resist rotational movement of male connection portion relative to the female connection portion, and
wherein in the release configuration, the pin is positioned within the slot.

2. The handle assembly of claim 1, wherein the first component comprises a spool configured to connect to a release suture which extends through the inner bore of the second component and is tied to the prosthetic implant.

3. The handle assembly of claim 1, wherein the second component comprises a spool configured to connect to a release suture which extends through a lumen of the first component and is tied to the prosthetic implant.

4. The handle assembly of claim 1, wherein the slot comprises an axial portion extending parallel to the axial axis and a circumferential portion connected to the axial portion and extending circumferentially about the axial axis, wherein the retaining portion is connected to a terminal end of the circumferential portion.

5. The handle assembly of claim 4, wherein the circumferential portion comprises a deflectable portion adjacent to the retaining portion such that the deflectable portion can be dilated when the pin is received in the deflectable portion.

6. The handle assembly of claim 5, wherein the female connection portion comprises at least one relief channel extending parallel to the circumferential portion and configured to allow the deflectable portion to axially dilate when the pin moves through the deflectable portion.

7. The handle assembly of claim 5, wherein the female connection portion comprises a deformable material around the deflectable portion such that when the pin moves through the deflectable portion, the pin can compress the deformable material and axially dilate the deflectable portion.

8. The handle assembly of claim 1, wherein the pin is a second pin and the slot is a second slot, wherein the male connection portion comprises a first pin extending radially outwardly from the main body, wherein the first pin is axially offset from the second pin by a predefined distance, wherein the female connection portion comprises a first slot configured to receive the first pin.

9. The handle assembly of claim 8, wherein the first slot comprises an axial portion extending parallel to the axial axis and a circumferential portion extending circumferentially about the axial axis, wherein the axial portion has an axial length equal to the predefined distance.

10. The handle assembly of claim 9, wherein the male connection portion comprises a third pin extending radially outwardly from the main body, wherein the female connection portion comprises a third slot configured to receive the third pin.

11. The handle assembly of claim 10, wherein the first pin and the third pin are positioned at circumferentially opposite sides of the second pin.

12. The handle assembly of claim 10, wherein the third slot comprises an axial portion extending parallel to the axial axis and a circumferential portion extending circumferentially about the axial axis, wherein the circumferential portion of the third slot is axially aligned with the circumferential portion of the first slot.

13. The handle assembly of claim 1, wherein the retaining portion comprises a radially recessed detent relative to the slot and the inner surface the female connection portion.

14. The handle assembly of claim 13, wherein the slot comprises a radial protrusion adjacent to the radially recessed detent.

15. The handle assembly of claim 14, wherein the radial protrusion is configured to be radially depressible such that the radial protrusion can be radially compressed to allow the pin to move across the radial protrusion.

16. The handle assembly of claim 14, wherein the pin is configured to be deformable such that the pin can be radially compressed when moving across the radial protrusion.

17. A handle assembly for a delivery apparatus configured to deliver a prosthetic implant, the handle assembly comprising:
a suture lock configured to connect to a release suture which is tied to the prosthetic implant; and
an adaptor comprising a lumen configured for the release suture to extend through and an end portion configured to be releasably connected to a connecting portion of the suture lock via a lock and release mechanism,
wherein the lock and release mechanism comprises a peg and a slot configured to receive the peg, wherein the slot comprises a deflectable portion and a retaining portion connected to the deflectable portion, wherein the retaining portion is configured to retain the peg, wherein the deflectable portion has a smaller diameter than the peg,
wherein rotation of the suture lock relative to the adaptor in a first direction is configured to move the peg through the deflectable portion and lock into the retaining portion such that the deflectable portion inhibits the peg from moving out of the retaining portion,
wherein rotation of the suture lock relative to the adaptor in a second direction opposite to the first direction is configured to move the peg out of the retaining portion through the deflectable portion.

18. The handle assembly of claim 17, wherein when connected, the connecting portion of the suture lock and the end portion of the adaptor are coaxial about an axial axis.

19. The handle assembly of claim 18, wherein the slot comprises a first slot portion extending parallel to the axial axis and a second slot portion connecting to the first slot portion at a corner and extending circumferentially about the axial axis, wherein the second slot portion comprises the deflectable portion and the retaining portion is located at a terminal end of the second slot portion.

20. A method of implanting a prosthetic implant, the method comprising:
assembling a handle assembly according to claim 1,
wherein the assembling comprises rotating the suture lock relative to the adaptor in the first direction so as to connect the suture lock to the adaptor.
